(19) United States
(12) Patent Application Publication
(10) Pub. No.: US 2010/0216649 A1
(43) Pub. Date: Aug. 26, 2010

(54) Methods for protein interaction determination

(76) Inventors: Steven C. Pruitt, Williamsville, NY (US); Alex R. Hastie, Munich (DE); Lawrence M. Mielnicki, Buffalo, NY (US)

Correspondence Address: HODGSON RUSS LLP;THE GUARANTY BUILDING, 140 PEARL STREET, SUITE 100, BUFFALO, NY, 14202-4040, US

(21) Appl. No.: 12/011,234

(22) Filed: Jan. 25, 2008

Related U.S. Application Data
(63) Continuation-in-part of application No. 10/842,741, filed on May 10, 2004, now Pat. No. 7,323,313.
(60) Provisional application No. 60/469,342, filed on May 9, 2003.
(60) Provisional application No. 60/977,923, filed on Oct. 5, 2007.

(51) Int. Cl. C40B 30/00; C12N 15/63

(52) U.S. Cl. 506/7; 435/320.1

(57) ABSTRACT

Provided are compositions and methods for identifying pairs of interacting proteins. The pair of plasmids is adapted for use in a modified two hybrid system wherein each plasmid comprises a recombinase recognition site. The method comprises the steps of providing cDNAs encoding test polypeptides, inserting the cDNAs into the first and second plasmids, recombining the first and second plasmids to obtain recombined plasmids, isolating and digesting the recombined plasmids to obtain cDNAs encoding pairs of interacting proteins, and determining the sequence of the digested fragments to determine pairs of interacting proteins.

This application claims priority to U.S. patent application Ser. No. 60/977,923, filed on Oct. 5, 2007, and is a continuation-in-part of U.S. patent application Ser. No. 10/842,741, filed on May 10, 2004, which in turn claims priority to U.S. patent application Ser. No. 60/469,342, filed on May 9, 2003, the disclosures of each of which are incorporated herein by reference.

This invention was supported by grant number GM68856 from the National Institutes of Health. The Government has certain rights in the invention.

### FIELD OF THE INVENTION

The present invention relates generally to the area of protein interactions and more particularly provides methods and compositions useful for rapid identification of protein interactions.

### BACKGROUND OF THE INVENTION

It is widely recognized that binding between proteins is central to virtually all biological processes. With several completed genome sequences as a frame work with which to interpret such interactions, several large scale projects have attempted to define protein interactions for all of the open reading frames of simple organisms including viruses, bacteria, yeast, Drosophila and C. elegans.

Although other methods of defining protein interactions are possible, the most highly developed method for genome-wide analysis is the original yeast two-hybrid system in which interactions are monitored by the induction of gene expression. This technology can be used in a variety of cell types, including mammalian cells.

Two hybrid analysis works by separating the DNA binding domain (DBD) and activation domain (AD) of a transcriptional activator by cloning their respective coding sequences into separate vectors. One or both DBD and AD coding regions are then fused to many different open reading frames (ORFs), typically from a cDNA library. In the case where the two hybrid system is used in yeast, the DBD and AD vectors can be introduced into the same cell by mating and using DBD and AD vectors that each includes a selectable marker.

If the proteins expressed from the ORFs physically interact, the two halves of the transcriptional activator are brought together and the function of the transcriptional activator is restored. The reconstituted transcriptional activator can then drive expression of a selectable marker, such as a nutritional marker. When the reporter gene is detected, the plasmids with the interacting DBD and AD can be isolated from yeast colonies and the interacting ORF's identified by DNA sequencing.

Large scale projects to define all of the interactions occurring between all of the ˜6,000 open reading frames in yeast have been accomplished using the yeast two hybrid system. However, application of this technology to mammalian genomes, which contain on the order of 10-fold greater complexity, is currently not feasible due to the exponentially greater number of potential interactions that must be scored. Thus, there is a need for an efficient method of identifying genome-wide protein interactions for organisms with complex protein interactions. The present invention meets this need by providing a modification of two-hybrid technology that permits the identification of many pairs of interacting proteins.

### SUMMARY OF THE INVENTION

The present invention provides a method for identifying a plurality of pairs of interacting proteins and plasmids for use in the method.

The invention provides a plasmid pair adapted for use in a modified two hybrid system wherein first plasmid comprises a coding sequence for a DNA binding domain of a transcription activator (the “DBD plasmid”) and the second plasmid comprises a coding sequence for a transcription activation domain of a transcription activator (the “AD plasmid”), and each plasmid further comprises a recombinase recognition site.

The method comprises the steps of providing cDNAs encoding test polypeptides, inserting the cDNAs into the first and second plasmids, recombining the first and second plasmids to obtain recombined plasmids, isolating and digesting the recombined plasmids, and determining the sequence of the digested fragments. The sequence of the digested fragments can be obtained by any suitable method, such as by high throughput sequencing techniques, such as “massively parallel pyrosequencing” described in Margulies, et al. (2005) Nature, 437, 376-380). Massively parallel pyrosequencing is suitable for whole genome sequencing in microfabricated high-density picolitre reactors. Other suitable techniques as will be recognized by those skilled in the art can also be used.

Alternatively, determining the sequence of the restriction fragments can be performed by ligating the restriction fragments to a universal adapter to provide a pool of digested fragments flanked by a universal adapter, selecting and amplifying desired sequences, forming concatamers from the amplified sequences, and sequencing the concatamers to determine the nucleotide sequences encoding a plurality of pairs of interacting proteins.

### BRIEF DESCRIPTION OF FIGURES

FIG. 1. is a graphical representation of one embodiment by which the generation of AD (left) and DBD (right) libraries in yeast by homologous recombination mediated gap repair can be achieved.

FIG. 2. is a graphical representation of one embodiment of a scheme for mating AD and DBD libraries. Schematics of the vectors (episomes) carried by the MAT-alpha-AD library (left) and MAT-a-DBD library (right) strains are shown as circles. A tamoxifen inducible Cre-recombinase gene, under the control of a DEX responsive element is present in the MAT-alpha strain is indicated as the boxed “CREmer”. Both strains carry Ura3 and His3 under the control of UAS(G) where only the Ura3 gene is shown and is indicated as the boxed “URA3”.

FIG. 3A-C. are a graphical depiction of recovery of linked cDNAs and compression of the sequence data that is identified through a modification of the MAGE technology.

FIG. 3A is a graphical representation of pairs of linked, double stranded cDNAs are shown as they appear in the recombined plasmid. “A” and “a” in the hatched boxes represent the first pair and “B” and “b” represent the second pair of cDNAs. Also shown are the MmeI recognition site (closed circle), the MmeI cleavage site (arrow), and the recombined Lox66/71 sites.

FIG. 3B is a graphical representation of the products of MmeI digestion after ligation of universal adapters (“UA”) comprising an XbaI restriction endonuclease.

FIG. 3C is a graphical representation of concatamers of XbaI digest fragments of the polynucleotides of FIG. 3B. cDNAs encoding interacting proteins flank lox sites and are separated from other pairs of interacting cDNAs by remaining adapter and XbaI sequences.

FIG. 4. A cloning vector (ClonTech pGADT7-Rec) and a representation of one embodiment of a cDNA library construction strategy is shown wherein cDNAs are prepared containing termini that are homologous to the insertion site in the vector and the vector introduced to yeast as a linear molecule in combination with the cDNAs for ligation by homologous recombination. This results insertion of a type II S restriction endonuclease cleavage site sequence element at the fusion point between the activation domain and the cDNAs by modifying the CDS III oligonucleotide to include the Type II S restriction enzyme.

FIG. 5A. is a graphical representation of a high copy number 2μ based two-hybrid AD fusion vector with lox71 sequence integrated adjacent the 3′ cDNA cloning site. Also shown are various selectable markers and “3′ cDNA homology” and “5′ cDNA homology” sites for homologous with cDNAs.

FIG. 5B is a graphical representation of a low copy number CEN based two-hybrid DBD fusion vector with lox66 sequence integrated adjacent the 3′ cDNA cloning site with additional features as described for FIG. 5A.

FIG. 5C is a graphical representation of one embodiment of a product of a stable site directed recombination between the AD and DBD plasmids resulting in cDNA cloning sites directly adjacent the doubly mutated lox66/71 sequence.

FIG. 5D is a representation of a Southern blot demonstrating in vivo Cre dependant recombination between lox66 and lox71 sequences adjacent the 3′ cDNA cloning site of Gal4 DNA binding domain (DBD) and Gal4 activation domain (AD) Y2H vectors. The figure represents a Southern blot probed with a fragment of the ampicillin resistance gene. Lane X is a size ladder, Lane 1 is empty, Lane 2 is each plasmid digested by HindIII (carrots). Lanes 3 and 4 are controls, Lanes 5 and 6 are DNA harvested from HEK 293 cells digested by HindIII that were transfected with 8 mg each of pBluescript and the two Y2H vectors depicted in FIGS. 5A and 5B (lane 5) and pPGKcre and the two Y2H vectors (lane 6). The band denoted by an asterisk is the product of Cre recombination that includes the ampicillin resistance gene. Lane 1 is 30 pg/ea of HindIII digested pGADT7lox71 and pCDlox66 to show the size of the unrecombined plasmids (carrots).

FIG. 6 panels A-C, provides a schematic depiction of a cloning strategy to produce yeast-two-hybrid libraries for use of a mouse protein (HoxA1) as a bait fusion protein for screening an E12.5 mouse embryo cDNA library. The prey vector (panel A) pGADt7lox71 includes an Adc1 promoter, GAL4 AD cDNA, a hemagglutinin epitope, a gap-repair cloning sequence which includes the lox71 sequence, the 2μ ori, an ampicillin resistance gene for bacterial selection, and the LEU2 gene for yeast selection. The figure also shows a cDNA molecule flanked by vector homology, an MmeI RE binding site, and a lox71 site. The bait vector (panel B) pCD.2lox66HoxA1 includes an Adc1 promoter, GAL4 DBD cDNA, a hemagglutinin epitope, the HoxA1 sequence, an ampicillin resistance gene for bacterial selection, a CEN sequence for low copy number replication, and a TRP1 gene for yeast selection. A bait vector pGADt7lox71 for library creation (panel C) includes an ADH1 promoter, GAL4 DBD cDNA, a cMyc epitope, a gap-repair cloning sequence that includes the lox66 sequence, the 2μ ori, a kanamycin resistance gene for bacterial selection, and the TRP1 gene for yeast selection. The sequences presented in FIG. 6 are provided as:, Panel A: left side sequence: SEQ ID NO:33; right side sequence: SEQ ID NO:34; Panel B: left side sequence: SEQ ID NO:35; right side sequence: SEQ ID NO:36; Panel C: left side sequence: SEQ ID NO:37; right side sequence: SEQ ID NO:38.

FIG. 7 provides a photographic representation of electrophoretically separated unrecombined and recombined vectors in the presence and absence of Cre recombinase, respectively. Specifically, a Southern blot of HindIII and PstI-digested yeast DNA probed with a GAL4 AD fragment is shown. Lane 1: Bait and prey vectors in yeast strain AH109 in the absence of Cre expression vector (AH109-pCDlox66, pGADt7lox71). Lane 2: Bait and prey vectors in yeast strain AH109 in the presence of Cre expression vector (AH109-pCDlox66, pGADt7lox71, pFA6a2pμ-Adc1Cre).

FIG. 8, panels A-E, provide schematic representation of steps performed in the use of bait and prey vectors to identify proteins that interact with HoxA1. Panel A) Bait and prey BI-Tag Y2H vectors with lox66 and lox71, respectively. Panel B) Cre induced recombination at lox sites causes linkage of the BI-Tag Y2H vectors. Panel C) MmeI digestion generates the BI-Tag-containing lox66/71 flanked by MmeI and 20-bp sequence tags. Panel D) NotI linkers are ligated to the BI-Tag, and PCR is performed. Panel E) After NotI digestion the BI-Tags are ligated to form concatamers.

FIG. 9, panels A-D, provide photographic representations of electrophoretically separated amplicons across interacting cDNAs that were generated using PCR with GAL4 DBD- and GAL4 AD-specific primers, and of restriction digest fragments of the PCR products. Panel A) PCR amplification (with primers that anneal to GAL4 AD and DBD cDNAs) across linked cDNAs and lox sequence of the HoxA1 Y2H positive colony DNA (bar). Panel B) MmeI digestion of the PCR product to produce the 86-bp BI-Tag (arrow). Panel C) Left lane: 160-bp PCR product that includes the BI-Tag and 40-bp linkers (arrow), Middle lane: 94-bp BI-Tag (arrow) generated by NotI digestion with NotI compatible overhangs for concatenation. Panel D) Amplicons of BI-Tag concatamer inserts in a cloning vector.

### DETAILED DESCRIPTION OF THE INVENTION

The present invention provides compositions and methods for determining the identity of pairs of interacting proteins. In one embodiment, a method is provided for determining the identity of a plurality of pairs of interacting proteins. A “pair of interacting proteins” comprises a first test protein and a second test protein, wherein the first and second test proteins interact with each other in a cell.

Overall, the method of the present invention can be represented by the following steps.

a) providing a library of test cDNAs in which protein-protein interactions are to be determined;

b) providing a first and a second plasmid adapted for the modified two hybrid system, wherein the first plasmid comprises the coding region of a binding domain of a transcription activator (DBD plasmid) and the second plasmid comprises the coding region of a transcription activation domain for the transcription activator (AD plasmid), and wherein both plasmids have elements for homologous recombination with cDNAs encoding the first and second test proteins, promoters for driving transcription of the inserted cDNAs, drug selection, nutritional selection, origins of replication and recombinase recognition sites;

c) inserting the cDNAs into the first and a second plasmids such that each plasmid has one cDNA inserted therein thereby creating a library of inserted first and second plasmids;

d) obtaining recombined plasmids by i) introducing a pair of a first and a second inserted plasmids into host cells to obtain recombined plasmids in the host cells or ii) introducing the first inserted plasmid into a host cell and the second inserted plasmid into another host cell and allowing mating of the two host cells

e) isolating and digesting the recombined plasmids to obtain from each recombined plasmid, a restriction fragment comprising a sequence from each of the two interacting proteins; and determining the sequence of the restriction fragments.

In one embodiment, the sequence of the restriction fragments can be performed by the optional steps of:

f) flanking each restriction fragment with a sequence for a universal adapter;

g) ligating the flanked restriction fragments to form concatamers, wherein the concatamers comprise from 5′ to 3′: universal adapter sequence, a first cDNA sequence encoding a first test protein, Type II S restriction enzyme recognition sequence, recombinase recognition sequence, Type II S restriction recognition sequence, and a second cDNA sequence encoding a second test protein, wherein the first and second cDNA sequences are from a single recombined plasmid; and

h) sequencing the concatamers to determine the identity of interacting proteins.

Alternatively, the sequence of the restriction digestion fragments can be performed by any suitable high throughput sequencing techniques. In one embodiment, the technique is performed by massively parallel pyrosequencing as described in Margulies, et al. (2005) Nature, 437, 376-380. Massively parallel sequencing services are commercially available, such as from 454 Life Sciences (Branford, Conn.).

Thus, the present invention provides a vector system and methods for establishing a comprehensive protein interaction map from a cDNA library by adapting two hybrid technologies to allow physical linkage of cDNAs encoding interacting proteins and to improve the efficiency of identifying interacting cDNA sequences by high throughput sequencing methods, such as massively parallel sequencing, or by adaptation of a modified serial analysis of gene expression (MAGE). The elements for MAGE are described in U.S. patent application Ser. No. 10/227,719, filed on Aug. 26, 2002, which is incorporated herein by reference and is discussed more fully below. The modified two hybrid system of the present invention generates physically linked cDNAs which encode interacting proteins and which can be concatamerized for efficient analysis by MAGE. The advantage of this approach is that it is possible to identify many pairs of interacting proteins from a single mixed pool of yeast, or other cell types appropriate for the two-hybrid system used, in which multiple, different, protein-protein interactions are represented. Additionally, the data compression technique MAGE has been adapted in the present invention to allow improved efficiency in a cDNA sequencing step.

The method comprises the step of ligating a cDNA library into each of a first and second set of plasmids and transforming the plasmids into cells. Methods of ligating cDNA libraries into plasmids are well known to those skilled in the art. For example, the cDNAs and plasmids can be digested by a restriction enzyme and ligated in vitro. Alternatively, the cDNA library can be generated with specially adapted 5′ and 3′ ends for use in a yeast cell wherein the cDNA library and a linearized plasmid can be inserted into the yeast cell and joined together by the homologous recombination system of the yeast cell.

According to the method of the invention, the first plasmid comprises a coding sequence for a DNA binding domain of a transcription activator (the “DBD plasmid”) and the second plasmid comprises a coding sequence for a transcription activation domain of a transcription activator (the “AD plasmid”), and each plasmid further comprises a recombinase recognition site. The DBD coding sequence is configured such that insertion of a cDNA into the DBD plasmid will result in the expression of a fusion of the DBD and a first test polypeptide encoded by the inserted cDNA. Similarly, the AD coding sequences are configured such that insertion of a cDNA into the AD plasmid will result in the expression of a fusion protein comprising the AD domain and a second test polypeptide encoded by the cDNA.

When a DBD and AD plasmid are in the same cell and their respective cDNAs encode test polypeptides that interact with each other, the interacting test polypeptides will bring into physical proximity their respective fused DBD and AD domains such that transcription of a selectable marker is driven from the promoter to which the DNA binding protein binds. In this way, cells having plasmid pairs comprising cDNAs that encode interacting test polypeptides can be selected for. The selection can be by means of a marker, wherein the expression of the marker permits the cell to be identified and/or survive. For example, the selectable marker can be a reporter gene, such as EGFP, an epitope allowing Ab selection, or a marker that permits the cell to survive, such as an auxotrophic marker or resistance to an otherwise toxic agent.

If cells comprising both the AD and DBD plasmids encoding interacting test polypeptides are present in the same cell, a recombinase acts to recombine the vectors at the recombinase recognition sites which results in the physical linkage of cDNAs encoding interacting test polypeptides. Physically linked cDNAs encoding interacting polypeptides are also referred to herein as “BI-Tags.”

The recombined plasmids can then be digested with a Type II S restriction enzyme to obtain BI-Tags and the resulting restriction fragments can be sequenced using any suitable method to determine the nucleotide sequences of cDNAs encoding pairs of interacting test polypeptides.

### Plasmids

The present invention accordingly provides a plasmid system comprising AD and DBD plasmids. In addition to the activation domain on the AD plasmid and the DBD domain on the DBD plasmid, each plasmid may comprise selectable markers such as antibiotic and/or nutritional markers, origins of replication, promoters, transcription terminators, a wild type or mutant recombinase recognition site, and cloning sites for insertion of cDNAs, as will be more fully described below.

Selectable markers for use in prokaryotic and eukaryotic systems are well known. For example, selectable markers for use in prokaryotes typically include genes conferring resistance to antibiotics such as ampicillin, kanomycin or tetracycline. For eukaryotes, neomycin (G418 or geneticin), gpt (mycophenolic acid), puromycin or hygromycin resistance genes are suitable examples of selectable markers. Genes encoding the gene product of auxotrophic markers (e.g., LEU2, URA3, HIS3, TRP1, ADE2, LYS2) are often used as selectable markers in yeast and are well known in the art. Further, dihydrofolate reductase marker genes permit selection with methotrexate in a variety of hosts.

Origins of replications included with the plasmids of the invention are considered to be sequences that enable the plasmids to replicate in one or more selected host cells independently of the host chromosomal DNA and include autonomously replicating sequences. Such sequences are well known for use in a variety of prokaryotes and eukaryotes. Examples of origins of replication for use in a plasmids in eukaryotic host cell include the 2 micron origin of replication, ARS1, ARS4, the combination of ARS1 and CEN3, and the combination of ARS4 and CEN6. Examples of origins of replication for use in plasmids in a prokaryotic cell include pBR322 and pUC.

Examples of promoters useful in practicing the present invention include any promoter that can drive the expression of a selectable marker. Preferable promoters are those that can be activated by a transcription activator comprising a DBD domain and a transcription AD, such as the VP16 or GAL4 promoters.

In one embodiment, an expression plasmid containing the AD or DBD domain is preferably a yeast vector such as pACT2 (Durfee et al., Genes Dev. 7, 555, 1993), pGADT7 (“Matchmaker Gal4 two hybrid system 3 and libraries user manual” 1999), Clontech PT3247-1, supplied by Clontech, Palo Alto, Calif.) or pCD2 (Mol. Cell. Biol., 3, 280 (1983), and plasmids derived from such yeast plasmids.

cDNA Libraries

cDNAs for insertion into the vectors of the present invention can be obtained by PCR amplification using well known techniques. In general, total RNA is isolated from cells according to well known methods and reverse transcriptase synthesized mRNA is generated using random priming for the first strand synthesis. Subsequent rounds of amplification are performed using standard PCR techniques.

In one embodiment of the invention, sequence fragments homologous to the sequences on the plasmid vector are added to the 5′ and 3′ ends of each cDNA in the RT-PCR and subsequent PCR amplifications. This can be achieved by using a pair of PCR primers that incorporate the added sequences. Any sequences can be added to the PCR primers according to those skilled in the art.

In one embodiment, SMARTIII and CDSIII primer sequences are modified to allow incorporation of a type II S restriction endonuclease cleavage site into the cDNAs. cDNA synthesis using the modified SMART primers can be performed with nanogram quantities of total RNA. The SMART system (i.e., see Clontech SMART PCR cDNA Library Construction Kit (July 1998) CLONTECHniques XIII (3):9-10) uses a modified random primer to prime synthesis of the first strand in a PCR reaction. When reverse transcriptase reaches the 5′ end of the mRNA a few additional nucleotides, primarily deoxycytidine, are added to the 3′ end of the cDNA.

SMART primers have an oligo(G) sequence at their 3′ ends. This oligo(g) hybridizes with the 3′ deoxycytidines, creating an extended PCR template. Reverse transcriptase (RT) then switches templates and continues replicating to the end of the oligonucleotide. The resulting single-stranded cDNA contains sequences that are complementary to the SMART primer. A SMART anchor sequence and the modified CDS primer derived sequences are then used as universal priming sites for end-to-end cDNA amplification by PCR. In one embodiment, long distance PCR (“LD-PCR”) can be performed using standard techniques which allows amplification of longer sequences.

Inserting cDNAs

cDNAs can be inserted into the vectors of the present invention using well known techniques. For example, the cDNAs and plasmids may be digested with restriction enzymes and ligated together in vitro.

Alternatively, the library of AD and DBA vectors of the present invention can be generated by exploiting the inherent ability of yeast cells to facilitate homologous recombination at a high efficiency. Yeasts such as Saccharomyces cerevisiae have inherent genetic machinery to carry out efficient homologous recombination. This mechanism is believed to benefit the yeast cells for chromosome repair purposes and is traditionally also called gap repair. By using homologous recombination in yeast, gene fragments such as cDNAs can be cloned into a plasmid vector without a ligation step. Accordingly, the linearized plasmids and the cDNAs are co-transformed into host cells, such as competent yeast cells. Recombinant clones may be selected based on survival of cells in a nutritional selection medium or based on other phenotypic markers. Either the linearized vector or the cDNA alone may be used as a control for determining the efficiency of recombination and transformation.

In one embodiment, the method comprises the step of transforming into a first set of yeast cells a library of cDNAs that are linear and double-stranded, and a first linearized plasmid, such as either the AD or DBD plasmid. Each of the cDNA sequences comprises a 5′- and 3′-flanking sequence at the ends of the cDNA sequence. The 5′- and 3′-flanking sequence of the cDNAs are sufficiently homologous to the 5′- and 3′-terminus sequences of the linearized plasmids to enable homologous recombination to occur. Using the same strategy, the linear and double-stranded cDNA sequences are transformed into a second set of yeast cells (either the AD or DBD) along with a second linearized plasmid.

Recombining the Plasmids by Cre-Mediated Linkage of cDNAs Encoding Interacting Proteins

In order to realize the potential of the present invention to identify many pairs of interacting proteins, it is necessary to recombine the first and second plasmids into a single plasmid. In one embodiment, the recombination was demonstrated by transfection of an AD plasmid and a DBD plasmid into a mammalian cell using standard techniques. Because the plasmids each comprise recombinase recognition sites, a recombinase is able to catalyze the recombination of the two plasmids into a recombined plasmid.

Any recombinase can be used for this purpose. A preferred recombinase is CRE recombinase. CRE is a 38-kDa product of the cre (cyclization recombination) gene of bacteriophage P1 and is a site-specific DNA recombinase of the Int family. CRE recognizes a 34-bp site on the P1 genome called loxP (locus of X-over of P1) and efficiently catalyzes reciprocal conservative DNA recombination between pairs of loxP sites. The loxP site consists of two 13-bp inverted repeats flanking an 8-bp nonpalindromic core region. CRE-mediated recombination between two directly repeated loxP sites results in excision of DNA between them as a covalently closed circle. Cre-mediated recombination between pairs of loxP sites in inverted orientation will result in inversion of the intervening DNA rather than excision. Breaking and joining of DNA is confined to discrete positions within the core region and proceeds on strand at a time by way of transient phophotyrosine DNA-protein linkage with the enzyme.

The CRE recombinase also recognizes a number of variant or mutant lox sites relative to the loxP sequence. Examples of these Cre recombination sites include, but are not limited to, the loxB, loxL and loxR sites which are found in the E. coli chromosome. Other variant lox sites include, but are not limited to, loxB, loxL, loxR, loxP3, loxP23, lox.DELTA.86, lox.DELTA.117, loxP511, and loxC2. In one embodiment of the invention, a pair of lox66 and lox71 sites can be used for in Cre-mediated recombination which results in mutant lox site resistant to recombination by Cre recombinase.

Examples of the non-CRE recombinases include, but are not limited to, site-specific recombinases include: att sites recognized by the Int recombinase of bacteriophage .lambda. (e.g. att1, att2, att3, attp, attB, attL, and attR), the FRT sites recognized by FLP recombinase of the 2μ plasmid of Saccharomyces cerevisiae, the recombination sites recognized by the resolvase family, and the recombination site recognized by transposase of Bacillus thruingiensis.

To physically link cDNAs encoding interacting proteins within the cell, a coding region for the recombinase is provided in the genome of the cell. A preferable recombinase is tamoxefin inducible Cre named CreMer under the control of a DEX inducible promoter comprising glucocorticoid response elements. The glucocorticoid response elements allow induction of CreMer expression to high levels on treatment with DEX but show very low basal levels of expression in its absence. Additionally the CreMer variant of Cre requires the presence of tamoxifen for activity. This dual control allow tights regulation and permits a high degree of control over the expression of Cre activity. Thus, when a cell comprising a coding region for CreMer and the DBD and AD plasmids of the present invention, administering DEX and tamoxifen to the cell will induce expression of CreMer and cause recombination of the vectors.

In another embodiment, the DBD and AD vectors of the invention are each present in yeast cells of the opposite sex. Because yeast has two sexes (a and α), the DBD and AD vectors can easily be introduced into the same yeast cell by mating DBD and AD vectors that each include a selectable marker. Accordingly, in one embodiment of the invention, a yeast cells comprising a DBD plasmid is mated to a yeast cell comprising a AD plasmid. The plasmids can be maintained separately from each other by the use of selectable markers, such as by nutritional selection. Upon mating and activation of CreMer supplied for example from a CreMer gene endogenous to one of the yeast strains, the AD and DBD plasmids will be recombined at their lox sites such that the lox sites will be present in between the cDNAs of the first and the second fusion test proteins. The recombined plasmids can be selected for by requiring the AD and DBD proteins to interact by way of their fusion test polypeptides and drive the expression of yet another selectable marker, such as a nutritional selectable marker. The most commonly used yeast markers include URA3, HIS3, LEU2, TRP1 and LYS2, which complement specific auxotrophic mutations in yeast, such as ura3-52, his3-D1, leu2-D1, trp1-D1 and lys2-201.

### Sequencing Recombined Vectors

A key to the ability of the present technology to provide a wide profile of protein-protein interactions is by permitting the efficient sequencing of cDNAs encoding the pairs of interacting proteins (i.e., the BI-tags). This can be accomplished using any suitable high throughput sequencing technique.

In one embodiment, a modified version of the Serial Amplification of Gene Expression technology in a high throughput format is employed. This technology is referred to as Modified SAGE technology (MAGE). Accordingly, the vectors may comprises the elements for the modified serial analysis of gene expression (MAGE), (described in U.S. patent application Ser. No. 10/227,719, filed on Aug. 26, 2002, incorporated herein by reference).

MAGE is a high throughput method for the identification of DNA sequences. The method depends on the incorporation of type II S endonuclease restriction (such as BsgI, BpmI, or MmeI) recognition sequences adjacent to inserted cDNAs. These type II S restriction endonucleases have the property that each cleaves DNA at a position 16, 20 or 21 nucleotides adjacent to its recognition sequence where the composition of the adjacent nucleotides is irrelevant. Using the example of BsgI and MmeI, the present invention takes advantage of this property to allow the amplification of up to 21 nucleotides of the cDNA sequence adjacent to the cDNA insertion site.

Following this, bits of unknown sequence information (BI-tags) can be identified because these are separated by repeats of a known sequence. In the present application, this may be accomplished by ligating the PCR products with the aid of a restriction endonuclease cleavage site present in both the universal primer and adjacent vector sequence. The ligated strings of sequence tags may be then cloned and sequenced. Thus, BI-tags representing pairs of interacting proteins can be identified from the sequences generated from the ligated PCR products.

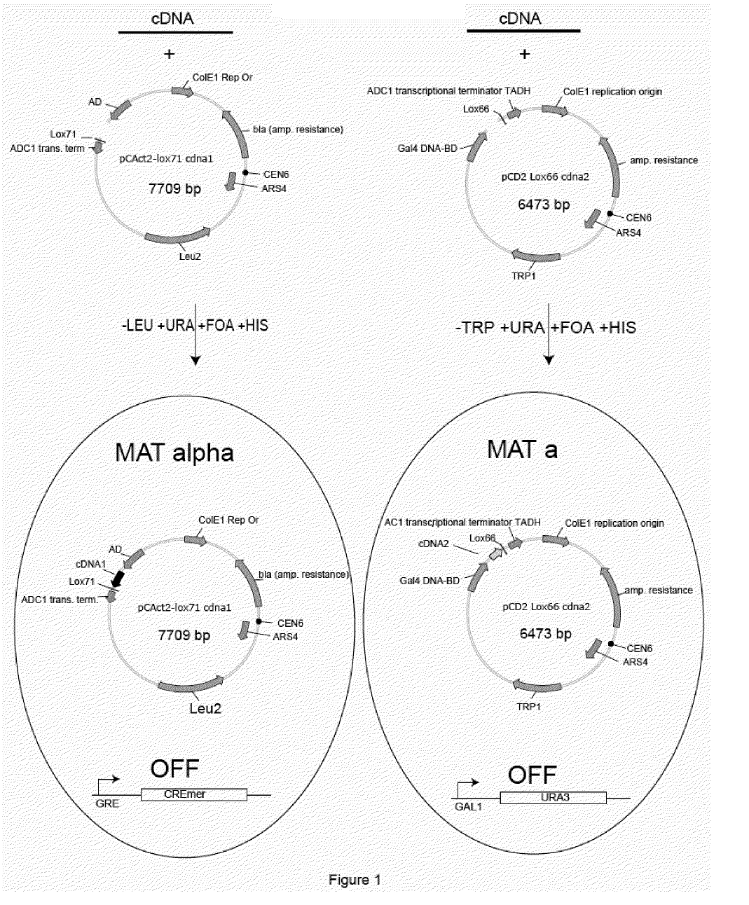

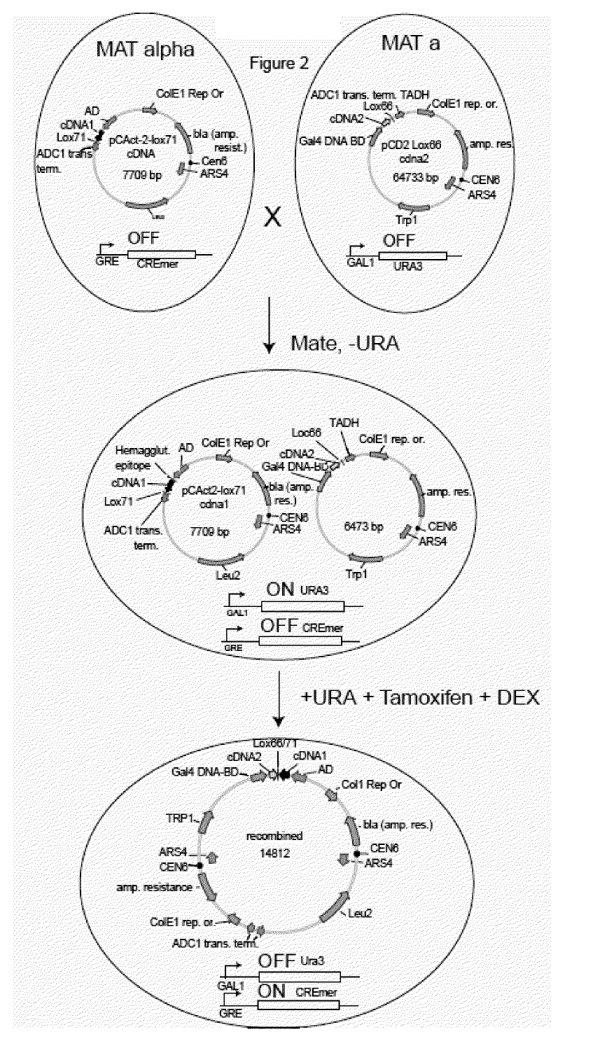

An illustrative overview of one embodiment of the invention utilizing yeast is shown in FIGS. 1-3. FIG. 1 illustrates the construction of the activation domain AD and binding domain DBD libraries in MAT-alpha and MAT-a strains of yeast. FIG. 2 illustrates mating of these strains and one embodiment for selection of interacting proteins by induction of recombination between plasmids comprising cDNAs encoding the interacting proteins. FIG. 2 shows graphical representations of particular embodiments of the plasmids carried by the MAT-alpha-AD library (left) and MAT-a-DBD library (right) shown as circles. A tamoxifen inducible Cre-recombinase gene under the control of a DEX responsive element is present in the MAT-alpha strain as indicated. Both strains carry Ura3 and His3 under the control of UAS(G) where only the Ura3 gene is shown. Strains are mated and selected for activation of the Ura3 and His1 genes mediated by two-hybrid interactions using SD-URA, -HIS dropout media. Following selection, physical linkage of the cDNAs encoding the interacting proteins may be accomplished by inducing CreMer expression with DEX and addition of tamoxifen. The orientation of the vector sequence can enable resolution of the recombined molecules, leaving the fused cDNAs on plasmid carrying the bacterial ori sequence, ampicillin resistance gene, a single centromeric sequence and either Trp or Leu (not shown). Recombination between the cDNAs will (or should) result in loss of Ura3 and His3 expression mediated by the interacting proteins. Selection for cells in which this has occurred is possible by growth on 5-FOA (not shown).

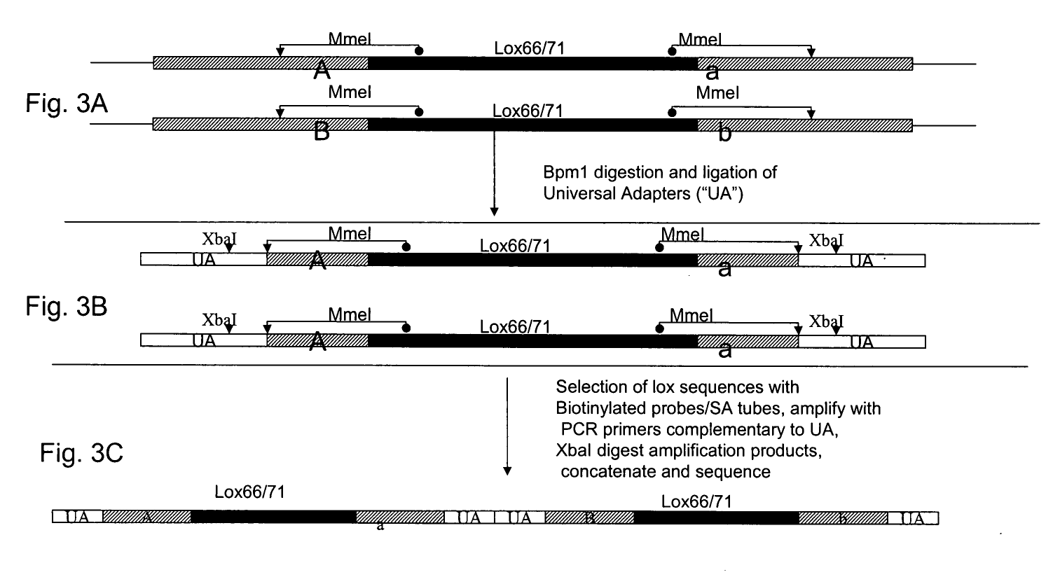

FIGS. 3A-C illustrate one embodiment for resolution of the sequence of the BI-tags by recovery of the linked cDNAs and compression of the sequence data with a modification of the MAGE technology. In FIG. 3A two pairs of linked, double stranded cDNAs are shown as they appear in the recombined plasmids. “A” and “a” in the hatched boxes represent the first pair and “B” and “b” represent the second pair of cDNAs. Also shown are the MmeI recognition site (closed circle), the BpmI cleavage site (arrow), and the recombined Lox66/71 sites.

FIG. 3B depicts the products of MmeI digestion after ligation of universal adapters (“UA”) comprising an XbaI restriction endonuclease. The cDNAs to be detected can be selected for in streptavidin (SA) tubes with biotinylated oligonucleotides that are complementary to the recombined lox sequences (not shown). The fragments depicted in FIG. 3B are amplified by PCR using primers complementary to the UA sequences. The amplification products are digested with XbaI and ligated together to form concatamers as shown in FIG. 3C.

As can be seen from in FIG. 3C, the cDNAs encoding interacting proteins can be determined because each cDNA of a pair is separated from its mate by an intervening lox sequence, and each pair of cDNAs is separated from other pairs by the UA sequence remaining after XbaI digestion.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitations.

### Example 1

This embodiment demonstrates the construction of a pair of plasmids useful for practicing the present invention in yeast. In this embodiment, the starting point for construction of the AD and DBD vectors were pCAct2 (AD vector) and pCD2 (DBD vector), which were obtained through the American Type Culture Collection (ATCC). These vectors are low copy number and contain CEN6 sequence elements. In this embodiment, two modifications to these vectors were made to prepare them for Cre mediated recombination to physically link the cDNAs they carry.

First, a region of pCAct2 carrying the ADC1 promoter, AD, site of cDNA insertion and transcription termination site is inverted relative to the remaining vector sequences. This is required to allow resolution of recombined plasmids in the final step of the selection as will be described more fully below. Second, lox sequences are inserted in both pCAct2 and pCD2. In this embodiment, pCAct2 received the half site mutant lox71 and pCD2 received the half site mutant lox66. Recombination between these lox sites generated a defective lox66/71 element that is no longer able to mediate efficient recombination and locks in the fusion between the cDNAs even in the continued presence of Cre.

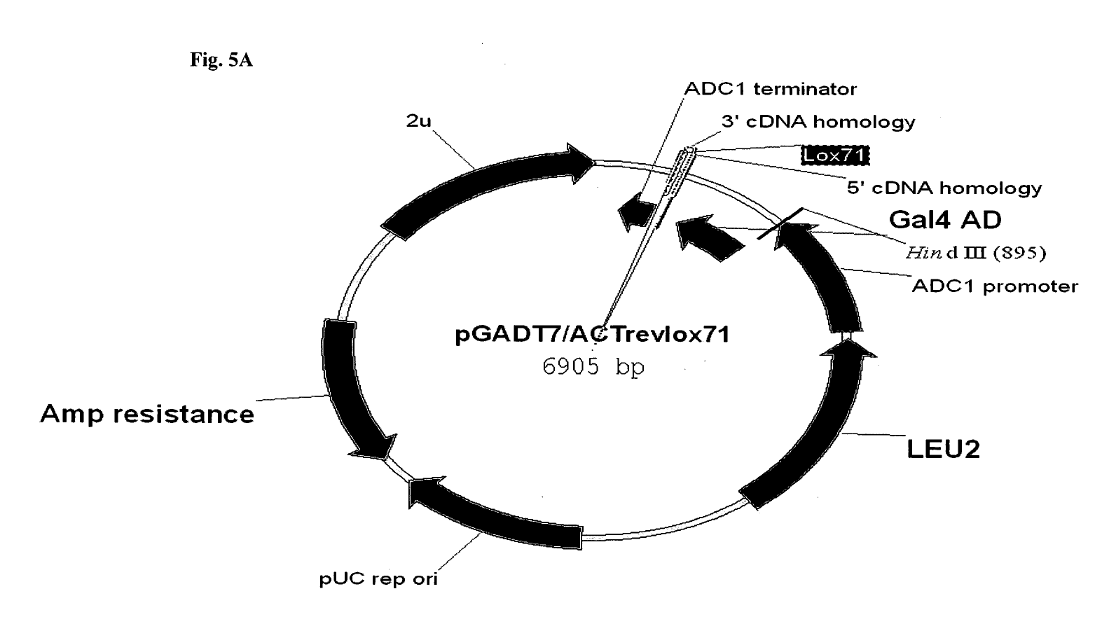

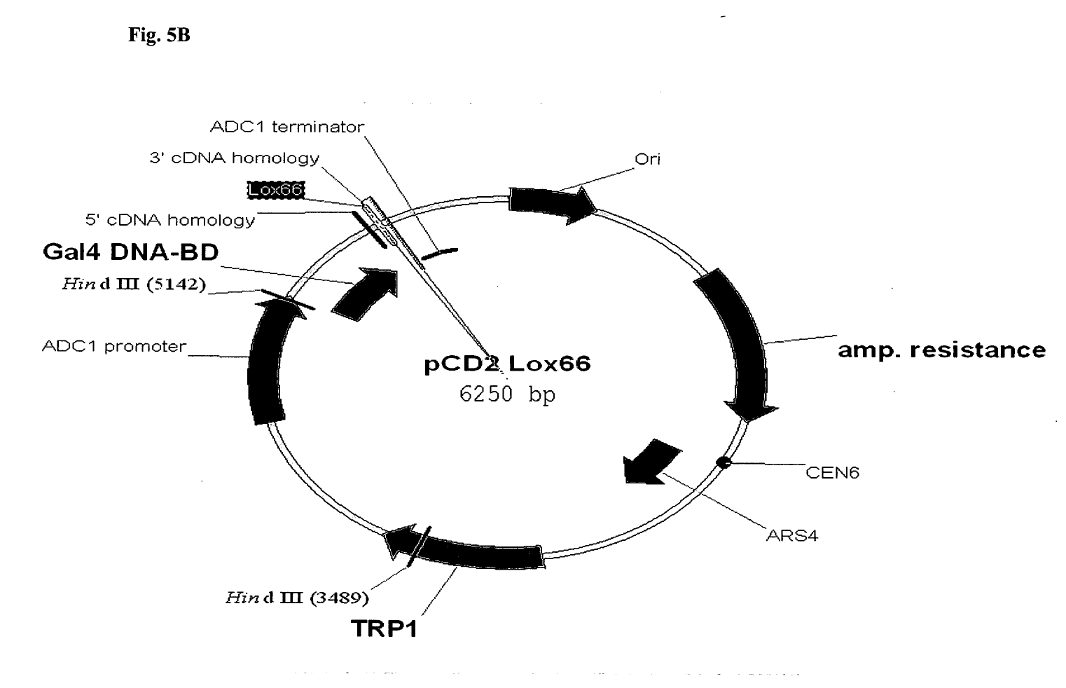

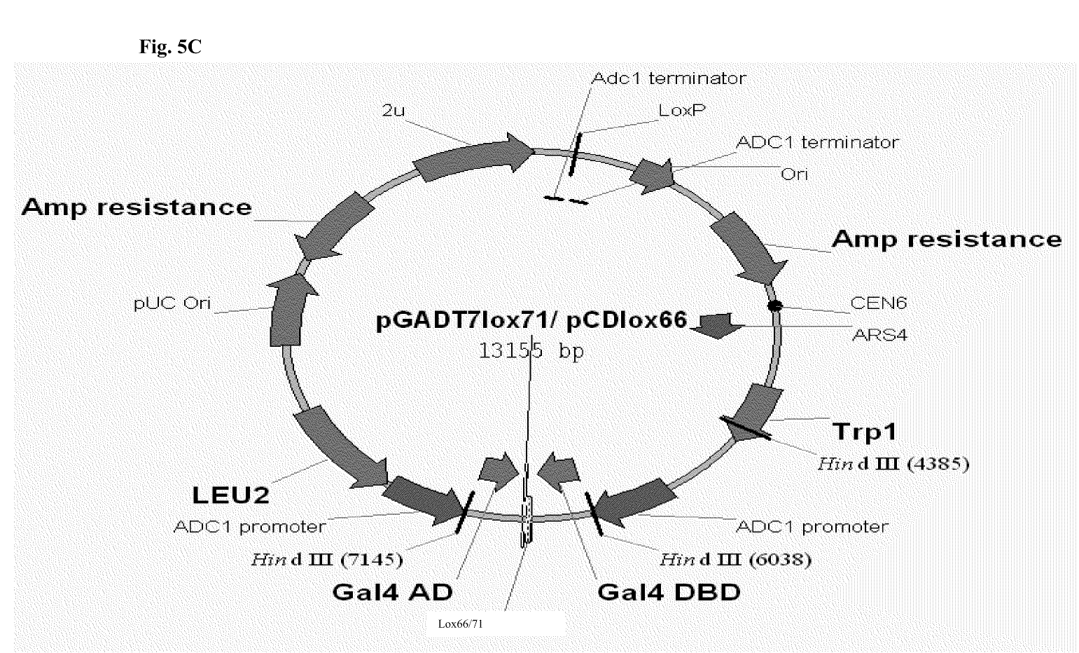

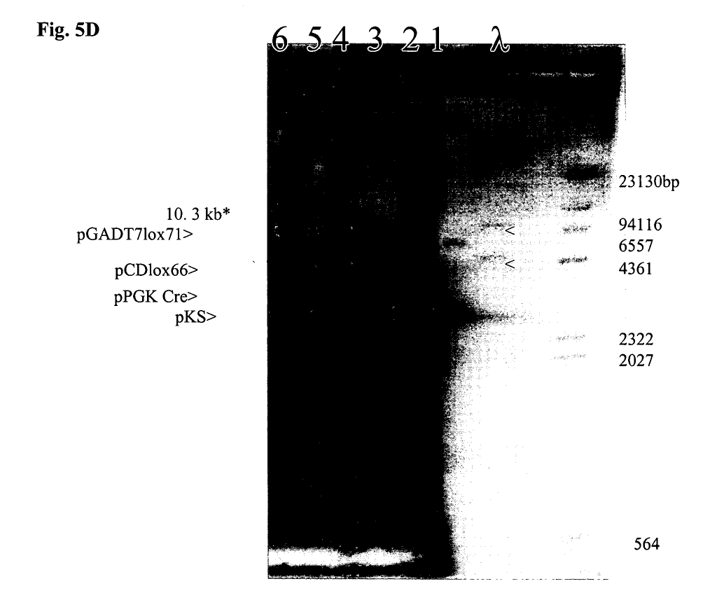

In another embodiment, a set of plasmids was also constructed that includes a high copy number 2 μm origin of replication. Shown in FIG. 5A is the pGADT7/ACTrevlox 71 plasmid which was constructed by removing the promoter, AD (or DBD), the cloning site, and the terminator from pGADT7rec and pGBKT7 (clontech) and replacing them with the ADC1 promoter, AD or BDB (as in FIG. 5B), the site of cDNA insertion, the lox71 sequence (or lox66 as in FIG. 5B) and transcription termination site from the CEN based plasmids described above.

### Example 2

This Example discloses one embodiment for the synthesis and incorporation of cDNAs into the AD and DBD plasmids described above by co-transfection of cDNAs containing the SMARTIII and CDSIII sequences with the AD and DBD plasmids.

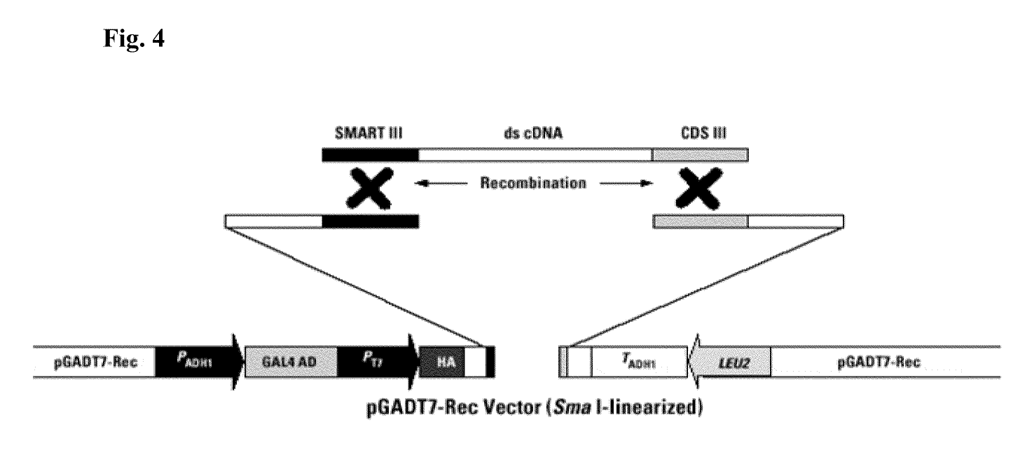

Outlined in FIG. 4 is the ClonTech® pGADT7-Rec vector and cloning strategy used in one embodiment of the invention. cDNAs were prepared containing termini that are homologous to the vector's insertion site and the yeast were transformed with linearized vector in combination with the cDNAs. Subsequent recombination at the homologous sequences generated the desired fusions and the re-circularization of the vector allows growth in yeast. This approach allows insertion of the BpmI, or MmeI, site needed subsequently for MAGE (as explained below) and requires only that the 3′ oligo sequence (equivalent to CDS III oligo shown in FIG. 4) is modified to include the BpmI, or MmeI, recognition sequences adjacent to the cDNA. The vector's homologous sequences are also modified to reflect those in the AD and DBD vectors described above.

In one embodiment, the primer sequences are:

(SEQ ID NO: 1)

pAct2 lox71 MAGE/6 Primer:
5′-GTATAGCATACATTATACGAACGGTAACCCTCTGAGCTGGAG-
NNNNNN-3′
Xba I Bpm I

(SEQ ID NO: 2)

PCD2 lox66 MAGE/6 Primer:
5′-CGTATAATGTATGCTATACGAACGGTACCCTCTGAGCTGGAG-
NNNNNN-3′
Xba I Bpm I

The Bpm I and Xba I sites are shown in bold. The 6 random nucleotides (N) are used to prime first strand cDNA synthesis and in many cases accurately represent the cDNA sequence.

In another embodiment, the primer sequences are:

(SEQ ID NO: 3)

Lox71 MmeI:
5′-TATAATGTATGCTATACGAACGGTAGGATCCAACNNNNNN-3′
MmeI

(SEQ ID NO: 4)

Lox66 MmeI:
5′-CATATCGTATGTAATATGCTTGCCATAGGTTGNNNNNN-3′
MmeI

The Mme I sites are shown in bold. The 6 random nucleotides (N) are used to prime first strand cDNA synthesis and in nearly all cases accurately represent the cDNA sequence.

Prior to cloning the cDNAs the cDNAs were normalized. The concentration of any specific message in the total population may vary over 3 to 4 orders of magnitude, hence the probability of finding interactions between two rare sequences would be low in the absence of a normalization step. A variety of methods have been described by which cDNAs can be normalized and any of these methods can be used in the present invention. In this embodiment, the normalization step was done by hybridization of cDNA to biotinylated driver cDNA, followed by removal of driver and abundant cDNA by streptavidin binding and phenol extraction. After normalization, the cDNAs were transfected into cells in conjunction with linearized AB and DBD plasmids to facilitate homologous recombination between the cDNAs and the plasmids.

The transformation efficiency of yeast using homologous recombination mediated gap repair is greater than 300,000 colonies per μg of starting vector. This efficiency is ample to allow generation of comprehensive cDNA libraries containing greater than 100,000 colonies. In this embodiment, the strains of yeast utilized take advantage of Ura3 selection from a Gal1 promoter to detect protein interactions. Ura3 expression can also be optionally counter-selected by the use of 5-fluoro-orotic acid (5-FOA, Boeke et al., 1984) which allows elimination of fusion proteins that auto-activate the Gal1 promoter in the absence of a dimerizing partner. Although a generally useful range of 5-FOA concentrations can be estimated from prior studies, titration of the concentration of 5-FOA against an aliquot of the transformed cells was performed where approximately 10,000 transformants were plated to a single 15 cm plate for each concentration in SD-URA media which also lacks either TRP or LEU depending on the vector. The same 5-FOA concentrations was used in parallel to test the effect on host cells in media containing URA, TRP and LEU. A concentration that has the maximum effect on suppressing growth of colonies from the cDNA libraries but minimal effect on the host cell was chosen for the remaining steps.

### Example 3

This example describes yeast cells having an endogenous CreMer gene for use with the present invention. The starting strains used for generating the CreMer expressing yeast strain were YD116 and YD119. These strains are both (ura3-52 his 3-200 leu2-trp1-901 can(R) gal4delta512 gal80delta338 lys2-801::UAS(G)-HIS3-lacZ ade2-101::GAL1-URA3) where YD116 is MAT-alpha and YD119 is MAT-a. To modify them for inducible Cre expression a tamoxifen inducible Cre variant (CreMer; Zhang et al., 1996) was inserted under the control of DEX inducible glucocorticoid response elements (Picard et al., 1990). This was accomplished by PCR based gene targeting using the pFA6a-kanMX6 module (Bahler et al., 1998) and selection in G418. Correct integration was confirmed by PCR. The glucocorticoid response elements allowed induction to high levels on treatment with DEX but show very low basal levels of expression in its absence. Additionally the CreMer variant of Cre requires the presence of tamoxifen for activity. This dual control allow tights regulation and permits a high degree of control over the expression of Cre activity. A strain of yeast of a particular sex harboring the CreMer gene and either a DBD or AD plasmid of the invention can be mated to a yeast of the opposite sex which harbors the complementary DBD or AD plasmid. In this way, activation of CreMer will catalyze recombination of the plasmids for sequencing analysis using the method of the present invention.

### Example 4

This Example demonstrates the mating of yeast cells wherein the opposite sexed cells harbor either DBD or AD plasmid such that mating the cells will provide cells with both DBD and AD plasmids. A comprehensive test of all interactions between the ˜100,000 cDNAs carried in the libraries generated above requires that 1×10¹⁰ diploid cells are generated. Optimized interaction-mating protocols have been developed that routinely allow mating efficiencies of 10% or greater (Soellick and Uhrig, 2001). These conditions are utilized here and require a low pH incubation of approximately 1×10⁸ cells/ml followed by seeding the cells to a filter at a density of 2×10⁷ cells/cm². Filters are transferred to agar and mating is allowed to occur for 4.5 hours prior to transfer to selection conditions. This protocol results in approximately 2×10⁶ zygotes/cm² of filter area. To achieve 1×10¹⁰ diploid cells requires the equivalent of 5,000 cm² of mating surface. Because a 15-cm filter allows approximately 176 cm² of surface, it is necessary to prepare approximately 30 such filters. Following mating, cells are removed from filters and pooled. Small aliquots are plated to SD-Leu, SD-Trp, SD-Leu-Trp to monitor the viability and mating efficiency. The remaining cells are plated to 15 cm plates in SD-Leu-Trp-Ura-His to select for interacting proteins. Based on an estimate of 300,000 potential interactions, each of 30 plates contain about 10,000 colonies, but the actual number of colonies is estimated and colonies are pooled.

### Example 5

This Example demonstrates that the plasmids of the present invention can be combined in vivo. As shown in FIG. 5D, transient transfection of the AB and DBD plasmids using standard techniques into HEK 293 cells depicted in FIGS. 5A and 5B above results in recombination of the plasmids.

FIG. 5D represents Cre dependant targeted recombination between lox66 and lox71 sequences adjacent the 3′ cDNA cloning site of Gal4 DNA binding domain (in the DBD plasmid) and Gal4 activation domain (in the AD plasmid) in vivo. Depicted is as Southern blot probed with a fragment of the ampicillin resistance gene. Lane 1 is empty, Lane 2 shows the two plasmids digested by HindIII (carrots). Lanes 3 and 4 are control reactions, and Lanes 5 and 6 show DNA harvested from HEK 293 cells. The cells were transfected with 8 mg each of pBluescript as a control and two plasmid vectors of the present invention (lane 5) and pPGKcre and the two Y2H vectors (lane 6). The DNA was isolated and digested by HindIII. The band denoted by an asterisk is the product of Cre recombination that includes the ampicillin resistance gene. This Example therefore demonstrates that the plasmids of the present invention are able to undergo Cre-mediated recombination in vivo.

### Example 6

This Example demonstrates how Cre-mediated linkage of cDNAs encoding interacting proteins can be performed within a yeast cell where the interaction is occurring. Approximately 1×10⁹ yeast cells in a total of 100 ml (1×10⁷ cells/ml) of the selected diploid cells can be inoculated to a liquid culture containing tamoxifen and DEX. In the absence of recombination, transcription of the Ura3 gene will continue because of the interaction of the AD and DBD cDNA fusion proteins at the Ura3 promoter. Ura, His, Trp and Leu may be present in this culture because recombination at the lox sites is expected to prevent expression of the fused cDNAs and resolution of the fusion plasmids through homologous recombination may lead to loss of either Trp or Leu resistance. Because the vectors used to construct the AD and DBD libraries carry a centromere and are low copy number, or in a situation where one or the other of the AD or DBD libraries carries a centromere and is present in low copy number, it may be useful to add FOA to the culture following sufficient time for Cre mediated recombination and the degradation of URA3 protein. This allows selection for cells in which lox sites have been recombined because, in the absence of recombination, transcription of the Ura3 gene will continue because of the interaction of the AD and DBD cDNA fusion proteins at the Ura3 promoter. The time required for efficient recombination and loss of URA3 activity can be determined empirically.

### Example 7

This Example illustrates a strategy by which recombined plasmids (episomes) can be recovered and linked cDNAs prepared for sequencing using the MAGE technique. Episomes, a portion of which comprising linked cDNAs A and a and B and b in the hatched boxes are as shown in FIG. 3A. These are recovered from yeast by standard techniques and used in a modified version of MAGE to extract sequence tag information from linked cDNAs. Linked sequence tags are referred to dimer-tags. Shown in FIG. 3B is the region of two episomes as prepared for linkage into a pool of linked cDNAs by ligation of a universal adapter (UA) which incorporates a restriction site (XbaI) into each MmeI fragment. Subsequent digestion with XbaI and concatamerization of the fragments results in linked pairs separated from each other by the lox66/71 sequence as shown in FIG. 3C.

To select specifically for the fragments containing the desired linked cDNA sequences, the intervening lox site is used as a hybridization probe. The ligation products are denatured and annealed to a 3′ biotinylated oligonucleotide homologous to this sequence. Use of a 3′ biotinylated probe prevents its participation in subsequent polymerization reactions. Hybrids are selected on streptavidin coated PCR tubes wherein the 3′ biotinylated oligonucleotide complementary to the lox sites hybridizes to the lox sites flanked by the cDNAs and thereby retains the cDNAs in the PCR tubes. Washing removes the large majority of contaminating sequences and following the wash step, oligonucleotides homologous to the top strand of the adapter sequence are used as PCR primers. PCR reaction products are digested with Not I for which there is a cleavage site present in adapter sequence. Each fragment results in a fragment containing the dimer-tag and 2×21 nucleotide long adapter fragments. These are electrophoresed on an acrylamide gel, the 86 by long fragment is recovered, ligated into concatamers and cloned into bacteria for sequencing. Any residual contaminating cDNA sequences that were not eliminated by the hybridization selection step will be further reduced in the population by size selection and are only a very minor contaminant, and such contaminants are easily recognized during sequencing.

A BD FACS-Vantage® with individual cell deposition capability is used to seed bacteria to microtiter wells for cloning. Standard high-throughput techniques are used to prepare plasmids for sequencing using protocols specific to suitable sequencing machines, such as Beckman® CEQ or Amersham® MegaBase 1000 capillary sequencers. Each sequence results in approximately 500-600 nt of useful sequence. Because each dimer-tag was 86 nucleotides in length, it was possible to identify an average of 5 interacting protein pairs from each sequence. This provides for cost-effective and comprehensive screening of protein interactions.

### Example 8

This Example demonstrates sequence tag analysis of cDNAs encoding AD test polypeptides that interact with Brn2 fused to the DBD of Gal4. These results were generated in yeast cells using DBD and AD plasmids wherein the AD cDNA library was created from poly A selected RNA from 9.5 day past coitus mouse embryos and in which a BpmI restriction enzyme site was incorporated adjacent the 3′ end of cDNA during synthesis.

Table 1 represents concatamers that were cloned and sequenced (tags are underlined, linker sequence is italicized, cloning vector sequence is bold). Table 2 represents the deconvoluted sequence tags from the SEQ ID NO:5 in Table 1, and Table 3 represents results from a BLAST search conducted on the identified sequence tags and representative cDNA GenBank accession numbers for the isolated cDNAs.

TABLE 1
(SEQ ID NO: 5)

ATCCCCCGGGCTGCAGGAATTCGATGCGATAATAACCACGGCCACCACTG
GAGGGATCCCTTGATCAGACACCACTGGAGCACGAGAAGAAGGAGCCACC
ACTGGAGCACGAGAAGAAGGAGCTCACCACTGGAGGGATCCCTTGATCAG
ACACCACTGGAGGGGGTCGGGACGGAGACACCACTGGAGGAGGGCACAGC
AGAAGCACCACTGGAGGGTGGGGACTTTCTCCCACCACTGGAGGGATCCC
TTGATCATACACCACTGGAGAGGGTCCCGATGCTGGCACCACTGGAGCCT
CGATCAGATCTGCCACCACTGGAGCACTAGAAAAAGAGGACACCACTGGA
GGAGGGCACAGCAGAAGCACCACTGGAGGGTGGGGACTTTCNTCCCACCA
CTGGAGTGCTCGTTAGAATATTCACCACTGGAGGGATCCCTTGATCANAC
ACNTNCTGGAGCGGACAGAGGANACNTCNACCACTGGAGCGGCAGGGGAA
CTTANCCCCACTTGGGACCACNANAAGNA

TABLE 2

| | | |
|---|---|---|
| 1. | CGATAATAACCACGGC | (SEQ ID NO: 6) |
| 2. | GGATCCCTTGATCAGA | (SEQ ID NO: 7) |
| 3. | CACGAGAAGAAGGAGC | (SEQ ID NO: 8) |
| 4. | CACGAGAAGAAGGAGCT | (SEQ ID NO: 9) |
| 5. | GGATCCCTTGATCAGA | (SEQ ID NO: 10) |
| 6. | GGGGTCGGGACGGAGA | (SEQ ID NO: 11) |
| 7. | GAGGGCACAGCAGAAG | (SEQ ID NO: 12) |
| 8. | GGTGGGGACTTTCTCC | (SEQ ID NO: 13) |
| 9. | GGATCCCTTGATCATA | (SEQ ID NO: 14) |
| 10. | AGGGTCCCGATGCTGG | (SEQ ID NO: 15) |
| 11. | CCTCGATCAGATCTGC | (SEQ ID NO: 16) |
| 12. | CACTAGAAAAAGAGGA | (SEQ ID NO: 17) |
| 13. | GAGGGCACAGCAGAAG | (SEQ ID NO: 18) |
| 14. | GGTGGGGACTTTCNTCC | (SEQ ID NO: 19) |
| 15. | TGCTCGTTAGAATATT | (SEQ ID NO: 20) |
| 16. | GGATCCCTTGATCANA | (SEQ ID NO: 21) |
| 17. | CGGACAGAGGANACNT | (SEQ ID NO: 22) |
| 18. | CGGCAGGGGAACTTAN | (SEQ ID NO: 23) |

TABLE 3
AGGGTCCCGATGCTGG (SEQ ID NO: 15)
gi|38084558|ref|XM_132640.2| Mus musculus empty
spiracles homolog 1 (Drosophila) (Emx 1), mRNA
GAGGGCACAGCAGAAG (SEQ ID NO: 12)
gi|25058121|gb|BC039041.1| Mus musculus zinc
finger protein 326, mRNA
GCAGATCTGATCGAGG (SEQ ID NO: 24)
gi|34447123|dbj|AB114630.1| Mus musculus CNR gene
for cadherin-related neuronal receptor

This Example therefore illustrates the ability of the method of the present invention to identify multiple cDNAs encoding proteins that interact to drive expression of a reporter gene.

### Example 9

This Example demonstrates the use of a mouse protein (HoxA1) as a bait fusion protein to screen for interaction partners (prey) in an E12.5 mouse embryo AD fusion protein cDNA library.

Plasmid Construction: The Lox71 sequence was added to the plasmid, pC-Act.2 (8), by adding double stranded oligonucleotides to create pC-Act.2lox71. The promoter driving the GAL4 AD coding sequence, lox71 and the transcription terminator were inverted with respect to the rest of the vector at AatII and Sad resulting in pC-Act.2lox71rev. pGADt7Lox71 was created by cloning the EcoRV and PvuII fragment of pC-Act.2revlox71 into SphI-, BsrGI-digested, blunt-ended pGADt7 (Clontech).

pCDlox66HoxA1 was cloned full length from pKS-HoxA1 (9), an MmeI site and lox66 sequence were included in the 3′ PCR primer and cloned into AvrII- and PstI-digested pCD.2 (8). pGBKt7lox66MmeI was constructed by ligating dsDNA containing lox66 and MmeI into pGBKt7 (Clontech) between EcoRI and SalI sites.

The SalI fragment of pCreERt2 (10) containing CreERt2 was cloned into pFA6a-KanMX6 (11) to make pFA6a-KanMX6-CreERt2. The 1600 by fragment of pGBKt7 (Clontech) containing the 2μ replication origin was cloned into SacII-digested pFA6a-KanMX6-CreERt2 to create pFA6a-KanMX6-CreERt2-4t. The Adc1 promoter was cloned from pCAct.2 as a NotI, ApaI fragment into pFA6a-KanMX6-CreERt2-2μ. The resulting vector is pFA6a-KanMX6-CreERt2-Adc1-2μ. Cre was inserted under the control of the Adc1 promoter by gap-repair cloning with KpnI-linearized pFA6a-KanMX6-CreERt2-Adc1-2μ and a PCR product templated by pBS185 (12). Finally, CreERt2 was removed by ApaI and AscI digestion and religation, resulting in the final construct, pFA6a2p.-Adc1Cre.

Yeast Strains and Library Construction: A+ RNA was purified from 12.5 dpc C57B1/6J mouse by the guanidine isothiocyanate (GITC) method and oligo dT-cellulose. The RNA was reverse transcribed with primers (for prey libraries, SMART pCAct: 5′-TGGCCATGGA CCTAGGCAGA TCTGATCAAG GGATCCGGG-3′ (SEQ ID NO:25) and CDS-52 lox71: 5′-GCTGCAGATA ACTTCGTATA ATGTATGCTA TACGAACGGT ATCCAACNNN NN-3′ (SEQ ID NO:26); for bait libraries, SMART pGBK47: 5′-GAGCAGAAGC TGATCTCAGA GGAGGACCTG CATATGGCCA TGGAGGG-3′ (SEQ ID NO:27) and CDS-54 lox66: 5′-GGCTGCAGCA TAACTTCGTA TAGCATACAT TATACGAACG GTATCCAACN NNNN-3′ (SEQ ID NO:28) adapted from the CLONTECH SMART cDNA synthesis protocol. Primary cDNA was amplified by PCR and cloned by gap-repair cloning with pGADt7Lox71 or pGBKt7lox66 vector and AH109 (Clontech) or YD116 for prey libraries or MATα strain YD119cre for bait libraries. Prey library transformants were selected for on medium lacking leucine. Bait libraries were selected for on SE (13) medium lacking tryptophan and containing G418. Negative selection was accomplished during library selection by adding 0.2% 5-FOA to the medium.

HoxA1 expressing bait strain: YD119Cre was transformed by pCDlox66HoxA1 and selected on SE medium containing G418 lacking tryptophan.

Yeast two-hybrid assay: The Y2H library E12.5-AH109-pGADt7lox71MmeI and YD119Cre-HoxA1 were mated, and were selected on SE medium (lacking leucine, tryptophan, adenine and histidine) and containing G418. Two-hybrid positive colonies from library screening were selected on SE medium (lacking leucine, tryptophan and uracil) containing G418.

Retesting: Retesting was performed by generating two PCR amplicons from each pick and recloning them by gap-repair cloning with pGBKt7lox66MmeI and pGADt7lox71 into YD119 and Yd116 respectively. Fusion proteins were tested for auto-activation by assaying for growth on medium lacking tryptophan and uricil or leucine and uricil. To retest interaction partners, corresponding interaction partner strains were mated and tested for URA3 gene expression. To test the ability of a protein to bind the GAL4 protein to activate the GAL4 responsive promoter, bait fusion proteins were mated with a strain carrying pGADt7lox71 and prey fusion proteins were mated with a strain carrying pGBKt7 and assayed for growth on SE medium -Trp, -Leu, and -Ura.

BI-Tag Y2H analysis: DNA was purified from the pool of two-hybrid positive colonies as described previously (14). PCR amplification of the linked cDNAs was performed with primers that anneal in the GAL4 DBD and GAL4 AD cDNA. The product was purified by phenol:chloroform:isoamyl alcohol (PCIA) extraction and EtOH precipitation. It was then digested by MmeI (NEB) and purified by 6% PAGE and the excised band was eluted into TE. Linkers (NotI linker t3: 5′-GCGGGATAGC GTGCCAGCGA GTGACGTTGC GGCCGCNN-3′ (SEQ ID NO:29), NotI linker b3: 5′-GCGGCCGCAA CGTCACTCGC TGGCACGCTA TCCCGC-3′ (SEQ ID NO:30); NotI linker t4: 5′-GGTATAGCCC GGCAGTTGCG CTGACGAGCA GCGGCCGCNN-3′ (SEQ ID NO:31), NotI linker b4: 5′-GCGGCCGCTG CTCGTCAGCG CAACTGCCGG GCTATACC-3′ (SEQ ID NO:32)) were ligated to the BI-Tags, then gel purified on PAGE followed by elution into TE. This DNA was used as template for PCR. The resultant 160 by band was purified by PCIA extraction and EtOH precipitation and digested by NotI, generating a 94-bp band. The 94-bp band was gel purified by PAGE, elution and EtOH precipitated. The pellet was dissolved in 6 ul of H₂O, and concatenation was performed in a 10 μl total volume with T4 DNA ligase (Invitrogen). DNA >500 by was purified from a 1.5% agarose gel and cloned into NotI-digested pBluescriptKS(+). Inserts were then amplified by PCR and sequenced.

Southern Blot: Southern blotting was performed using yeast total DNA prepared as described previously (14) and digested by PstI and HindIII The probe was a HindIII, EcoRI fragment of pC-Act.2 which contains the GAL4 AD cDNA.

Implementation of the materials and methods set forth above in this Example resulted in vector construction and Cre mediated recombination between AD and DBD yeast two-hybrid vectors in the following in vivo embodiment.

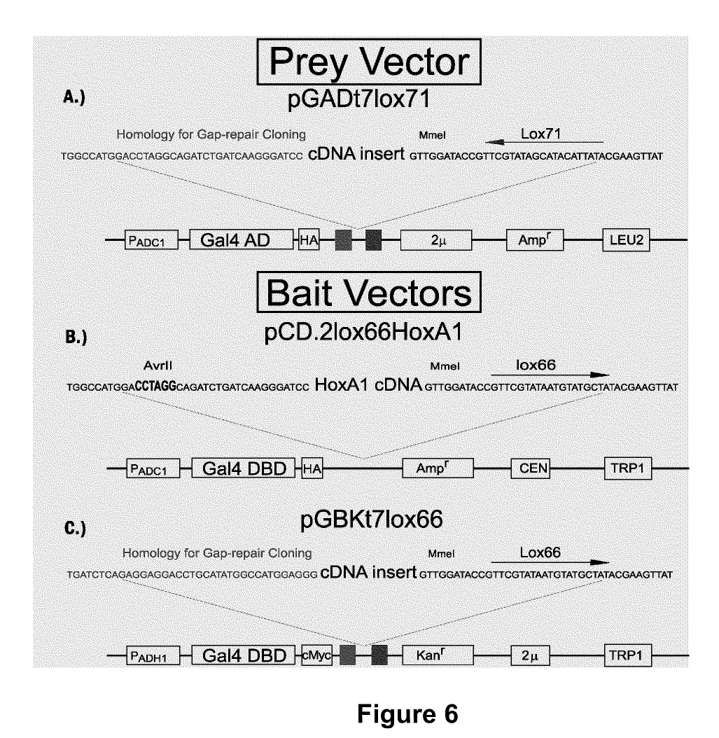

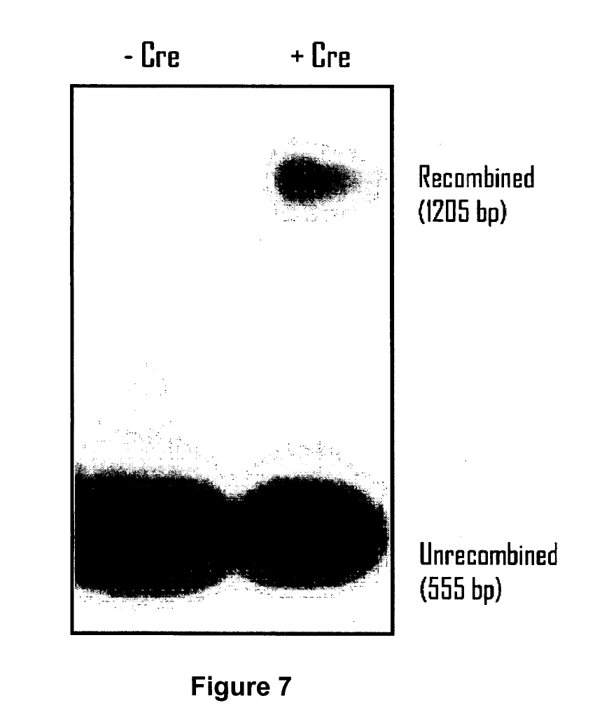

To produce yeast-two-hybrid libraries configured for use in the BI-Tag Y2H method in this Example, vectors were modified to contain mutant lox sequences adjacent to the 3′ end of the cDNA insertion site to obtain Bait and Prey vectors, as schematically depicted in FIG. 6. The vectors were transformed into yeast in the presence or absence of the Cre expression vector, pFA6a2g-Adc1Cre, and assessed for recombination (FIG. 7). As can be seen from FIG. 7, recombination occurs between lox71 AD vectors and lox66 DBD vectors in the presence of Cre, while no recombination is detected in its absence. It also should be noted that recombination between lox66 and lox71 creates a loxP site in addition to the lox66/71 site and that the loxP site will recombine with other lox sites forming higher order plasmids. These molecules are likely not stable and were not assayed for in the southern blot or in downstream applications (e.g. BI-Tag purification).

Library Preparation and Screening

To generate a library of cDNAs in the BI-Tag activation-domain vector, pGADt7lox71, cDNAs were prepared using poly-A positive RNA isolated from E12.5 day embryos. First strand synthesis was conducted by random priming with a primer containing five random nucleotides at the 3′ end, followed by an MmeI restriction enzyme site and ˜30 nts of vector-homologous sequence. MMLV reverse transcriptase was utilized to generate the first-strand cDNA. This enzyme has the property of incorporating several 3′ non-templated C residues following completion of first-strand synthesis. Second-strand cDNA synthesis was then accomplished by SMART technology (Clontech), which takes advantage of these C residues to prime second-strand synthesis using a second-strand primer that contains three G′ s at its 3′ end. Additionally, the 5′ end of the second-strand primer is homologous to the vector. These steps result in cDNAs with an MmeI site adjacent to the gene-specific DNA sequence and flanked by vector-homologous sequence that can be used for PCR amplification and gap-repair cloning in yeast. The MmeI site is used in subsequent steps to generate 20-bp tags for cDNA identification.

cDNAs prepared as above were used in gap-repair cloning in AH109 yeast (Clontech) with the pGADt7Lox71 vector to generate a library of 2.1×10⁶ total individual transformants and an average insert size of ˜500 by (called E12.5-AH109-pGADt7lox71MmeI). An insert size of 500 by will produce multiple fragments from most genes, which is anticipated to be advantageous since it has been shown that random-primed libraries detected valid two-hybrid interactions that were not seen when using full length ORFs (17).

A full length HoxA1 gene was cloned into pCDlox66, a CEN-based vector, as a GAL4 DBD fusion protein. CEN-based vectors are carried by yeast in one to three copies per cell, which eliminates the toxicity observed for some fusion proteins when they are expressed at high levels (8). The bait vector, pCDlox66HoxA1 was transformed into the Y2H yeast strain YD119Cre, which carries a plasmid that expresses Cre. This line was mated with the E12.5 library and selected for two-hybrid interactions, resulting in ˜1000 colonies.

### Comparison of Interaction Partners Identified by Individual Clone Analysis and BI-Tag Methodologies.

For comparison with the BI-Tag method, the library of clones selected for HoxA1 interactions was first characterized using standard methods. Eighteen individual Y2H positive fusion proteins were identified using a PCR based strategy similar to that described previously (2), and BLAST searches of NCBI's nucleotide database (Table 4). Table 1 is based on a comparison of BI-Tag and traditional Y2H analysis, HoxA1 screen: In the first column is a list of interaction partners that were identified by the BI-Tag method. The second column shows the number of times that BI-Tags were sequenced for each cDNA. The third column is the names of the cDNAs that were identified by traditional analysis followed by the number of times that each was identified.

TABLE 4

| BI-Tag IDs | # | Individual IDs | # |
|---|---|---|---|
| Uhrf1 | 36 | Uhrf1 | 2 |
| Lamc1 | 12 | Lamc1 | 4 |
| Col4a2 | 5 | Col4a2 | 3 |
| Hand2 | 10 | Sema3G | 4 |
| Psmd7 | 9 | Sdhb | 2 |
| eIF3s3 (or similar | 7 | Atp2a2 | 1 |
| to eIF3s3) | | | |
| Mtvr2 | 5 | Cpox | 1 |
| Gnb2 | 4 | Snrp1c | 1 |
| Sh3bp1 | 2 | | |
| Anapc7 | 1 | | |
| Arih2 | 1 | | |
| HnrnpA1* | 1 | | |
| Rprc1 | 1 | | |
| Pfn1 | 1 | | |
| Tubb5 | 1 | | |
| total | 95 | total | 18 |

*100% match at other non-gene genomic location

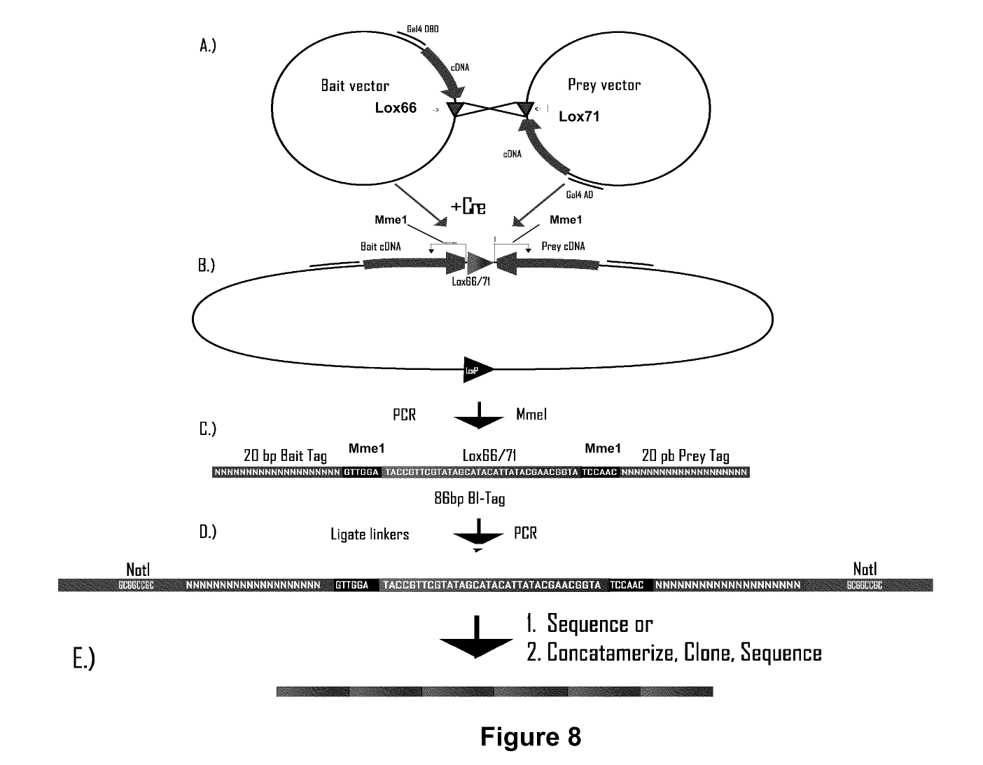

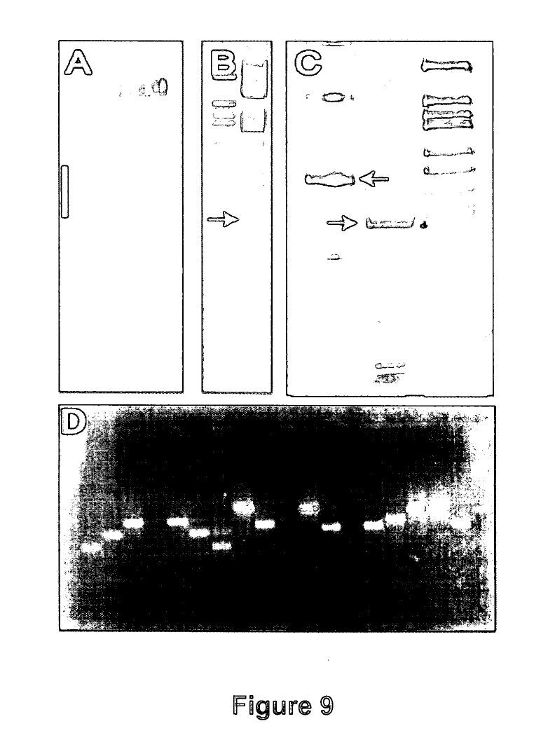

The BI-Tag method was then used (diagrammed in FIG. 8). First, DNA, which includes recombined plasmid DNA (FIG. 8a and b), was isolated from a pool of the ˜1,000-colony HoxA1 interaction library. Amplicons across interacting cDNAs were generated using PCR with GAL4 DBD- and GAL4 AD-specific primers (FIG. 8c). This reaction resulted in DNA fragments ranging from 1500 by and larger in length when assessed on a 1% agarose gel (FIG. 9a). Each amplicon includes the HoxA1-DBD fusion cDNA, an MmeI site, the lox66/71 double mutant recombination product, a second MmeI site, and the interacting AD-cDNA fusion. MmeI digestion was then used to excise an ˜86-bp fragment from each amplicon (FIG. 8c). These fragments are visible on a PAGE gel (FIG. 9b). These DNA fragments contain lox66/71 flanked by MmeI sites and the 19-21 bps BI-Tags used to identify the two interaction partners. Linkers with NotI cleavage sites were ligated to each end and used as primer binding sites in PCR amplification (FIGS. 8d and 8c, left lane). NotI digestion results in ˜94 by fragments, with complementary overhangs for concatenation, are gel purified by 6% PAGE (FIG. 9c, lane center lane). Purified DNA was ligated (FIG. 8e), and the resulting concatamers >500 bp were purified and cloned into a NotI-digested cloning vector. FIG. 9d shows amplicons across concatenated BI-Tags with size distributions between ˜300 and 700 bp. DNAs recovered from the clones were sequenced.

Unexpectedly, we found that in all but one case, all of the BI-Tags in each vector were orientated in the same direction. That is that the bait (HoxA1) tag was always on the left of the prey tag or vice-versa. BI-Tags were expected to have no preferred orientation within the cloning vector since each one has a NotI site on each end, which allows concatenation. This head to tail orientation could be a result of homologous recombination and/or hairpin formation within the bacteria during the BI-Tag cloning step which either deletes sequence or causes a selection against these clones.

BI-Tags were identified from sequence data by BLAST of the NCBI nucleotide database. A total of 95 tags representing 15 different genes were identified. A comparison of putative HoxA1 interacting proteins identified in the BI-Tag analysis described here with results from traditional individual clone analysis is shown in table 1.

### Example 10

This Example demonstrates DNA binding domain fusion protein library construction and screening for interacting partners in an activation domain library.

A bait library was constructed similar to the E12.5 prey library described above in Example 9, using pGBKt7lox66MmeI and YD119cre. Additionally, the medium contained 0.2% 5-FOA which is used to select against the presence of auto-activating bait fusion proteins (8). Previous studies have shown that ˜4-20% of all DBD-cDNA fusion proteins are auto-activating (2, 4, 6), i.e., are able to activate a GAL4 responsive promoter in the absence of a prey fusion protein. The resultant library, E12.5-YD119cre-pGBKt7lox66MmeI, had 5×10⁵ independent transformants.

A prey library was prepared as previously except the strain YD116 was used for negative selection against auto-activating prey fusion proteins. This library, E12.5-YD116-pGADt7lox71MmeI, contains 3×10⁵ transformants.

The libraries were mated and selected on two-hybrid selection medium (SE-Leu, -Trp, -Ura, 200 μg/ml G418). Thirty of these colonies were picked to a new plate, subjected to standard PCR amplification of cDNA inserts and sequenced for the identification of interaction partners. The result of this analysis is summarized in table 2, which presents data from a BI-Tag and traditional Y2H analysis, library by library screen: In the two columns are a list of interaction partners that were identified by the BI-Tag method. The third and fourth columns show the names of the cDNAs that were identified by traditional analysis. The numbers in superscript are the number of times a protein pair was found, the 1^{st} number is total times, and 2nd number is pairs with unique junctions (ensuring that it is from a unique clone).A total of seventeen different proteins were found from 54 cDNAs that were successfully sequenced. Based on analysis of the sequences we found that a bias was present in the cDNA synthesis step of the SMART library creation protocol that we used which significantly limited the diversity of bait and prey libraries. The SMART primer failed to prime correctly at the cytosine nucleotides added at the end of first strand by MMLV reverse transcriptase's terminal deoxycytodine transferase activity. Rather, priming occurred at short regions of homology (˜8-15 bps) within cDNA molecules resulting in inclusion of only a subset of cDNAs within the library.

TABLE 5

| BI-Tag IDs | | Individual IDs | |
|---|---|---|---|
| DBD cDNA | AD cDNA | DBD cDNA | AD cDNA |
| Arid1a | Nνc2^{2,1} | Arid1a | Nνc2² |
| Arid1a | Pcbp3^{13,11} | Arid1a | Pcbp3³ |
| Sf1 | Ttll12^{5,2} | Sf1 | Ttll12² |
| Sf1 | Pcbp3 | Sf1 | Pcbp3 |
| Sf1 | Dpysl2^{2,1} | Sf1 | Dpysl2 |
| Sf1 | Tubb5 | Sf1 | Tubb5 |
| 3100004P22Rik | Pcbp4 | Sf1 | Falz² |
| 4921511K06Rik | Mast2 | Aprt | Npc2 |
| Arid1a | Bnc2 | Arid1a | Khsrp |
| Arid1a | D530005L17Rik^{4,3} | Arid1a | Ttll12 |
| Arid1a | Itsn2 | Arid1a | Prmt7 |
| Arid1a | Numa1 | Arid1a | Ucp2 |
| Arid1a | Pcbp4^{3,1} | Arid1a | Dpys12 |
| Arid1a | Pfn1^{3,3} | Cugbp1 | Pcbp3 |
| Arid1a | Tubb5^{10,5} | Hmmr | Npc2 |
| Arid1a | Tubgcp2 | Rai17 | Npc2 |
| Arid1a | Ubl4 | Rai17 | Tln1 |
| BC021381 | Cfl1 | Sf1 | fblimp1 |
| Gm1302 | Zfp219^{2,1} | | |
| H2afv | Anxa6^{6,1} | | |
| Mrpl17 | Pfn1 | | |
| Myst4 | Pcbp3 | | |
| Ncoa2 | Atn1 | | |
| Palm | Fkbp8 | | |
| Plagl1 | Pcbp3 | | |
| Psmd8 | Ap2m1 | | |
| Rai17 | Ttll12^{2,2} | | |
| Sf1 | Hnrpab^{4,1} | | |
| Sf1 | Npc2 | | |
| Sf1 | Numa1 | | |
| Sf1 | Pcbp4 | | |
| Sf1 | Upc2 | | |
| Ss18 | Col1a1 | | |
| Ss18 | D530005L17Rik^{2,1} | | |
| Ss18 | Pcbp3^{2,1} | | |
| Ss18 | Scarb1 | | |
| Ss18 | Tubb5^{2,1} | | |
| Vim | Khsrp | | |

All of the colonies were collected, pooled and processed as previously for BI-Tag identification with the exception that BI-Tags were not concatenated. The results of this analysis are summarized in Table 5. From a total of 83 BI-Tags that were sequenced and contained one bait cDNA and one prey cDNA, each in the correct orientation, we found a total of 61 unique cDNA pairs which can be collapsed into 39 protein pairs.

In order to further characterize this library of Y2H positive interaction partners, we have subjected the 30 picks that have been identified by individual sequencing to several different tests. These picks include protein pairs which were also identified by the BI-Tag method. They were all retested for two-hybrid interactions (in the absence of cre), tested for auto-activation, and tested for their ability to bind to the GAL4 protein. Of the ten protein pairs which repeated in the retest experiments six were also found in the BI-Tag data (Sf1:Tt1112 twice in retest data, Sf1:Dpys12, Arid1a:Pcbp3, Sf1:Pcbp3 and Sf1:Tubb5). Also in the retest data there were two protein pairs which retested positively but the cDNAs were unable to be identified based on individual sequence reads because the PCR product was a doublet. Two retested protein pairs were not found in the BI-tag set (Cugbp1:Pcbp3 and Rai17:T1n1). Of the protein pairs which did not pass the retest, some failed to activate the two-hybrid promoter and other fusion proteins were able to activate the reporter in the absence of an interaction partner (by autoactivation or by binding the complementing Gal4 fragment). It should be noted that the Arid1a:Pcbp3 interaction retested positive only one time and failed two other times to retest which may indicate that one or both proteins may be somewhat promiscuous. Other high-throughput Y2H screening projects have reported that 55% (7), and about 20% (2) of first round two-hybrid positive protein pairs were reproducible. In all, we found that ten of twenty-three interactions (43%) retested successfully and many of these protein pairs were also found in the BI-Tag data set.

In high throughput Y2H interaction testing, multiple positive results with the same protein pair increases the likelihood that that protein pair represents a true interaction and is usually used as a criteria for confidence scoring of data (2, 3, 4, and others). Protein pairs from BI-Tag Y2H are not easily recovered for retesting but they can occur multiple times in a dataset and this criterion can be used as a surrogate for a retest. In this Example, there were 14 protein pairs which were identified by the BI-Tag method multiple times and three of these were retested, Sf1:Tt1112 and Sf1:Dpys12 had positive retests and Arid1a:Pcbp3 had a positive retest one of three times. Taken together, two of three protein pairs which were identified multiple times by BI-Tag Y2H were confirmed by retesting. This supports the notion that protein pairs found multiple times by BI-Tag Y2H are of higher confidence.

As shown in Table 5, one third of the individually identified pairs were also identified by the BI-Tag method, including five protein pairs that were shown to retest successfully. Additionally, all but one of the individually identified protein pairs that were identified multiple times were also identified by BI-Tag Y2H and the one case in which the protein pair was not represented (Sf1/Falz) could be explained by the occurrence of a mutation in the MmeI site.

Thus, the foregoing provides examples of Y2H interaction screening using Cre-mediated recombination to physically link, within the yeast cell, cDNAs encoding interacting bait and prey proteins as developed and tested using mouse HoxA1 as the bait protein for interaction partners in a library and by performing a library by library screen with mouse proteins.

### Efficiency of BI-Tag Y2H in Defining HoxA1 Interacting Proteins

The BI-Tag screen conducted in the present invention provides an illustration of creating physical linkage between interacting bait and prey cDNAs by using Cre-mediated recombination. Additionally, sequence tags of 19-21 nucleotides generated using MmeI as shown in FIG. 9 were sufficient to identify 95 of the 97 tags that localized within genes to either a unique gene (14 cases) or one of two closely related family members (2 cases). Comparison of the spectrum of prey molecules identified by individual sequencing from selected clones with those identified from BI-Tags showed that many of the more abundant clones (Uhrf1, Lamc1, and Co14a2-b) were identified in both data sets. However, a number of cDNAs were identified only in the BI-Tag, 13, or individual clone, 6 data sets, suggesting that each method may incorporate steps that result in a bias to the clones that are represented. By concatenating BI-Tags we were able to clone and sequence an average of 5 BI-Tags per sequencing run, resulting in a 5 fold reduction in sequencing requirements when one bait is used and prey proteins are identified. However, the unexpected head to tail orientation of all of the BI-Tags in these particular examples suggests the possibility that recombination is occurring within concatamers during amplification in bacteria.

The most prominent interaction partner identified in this screen (in both Bi-tag and individual clone datasets) is Uhrf1 (also referred to as Np95, ICBP90). Uhrf1 as well as several other potential interaction partners identified in the HoxA1 screen have been shown to be involved in the ubiquitin-proteasome pathways (Arih2, Psmd7, Anapc7). The ubiquitin-proteasome system has been shown to regulate transcription by several different mechanisms involving histone ubiquitination, transcription factor degradation or transcription activation by the proteasome in a ubiquitin dependant manner (for a review see 19). Uhrf1, Arih1 and the APC/C (of which Anapc7 is a component) have E3 ubiquitin ligase activity and Uhrf1 has been shown to ubiquitinate histones (20). Some of the other interactions are consistent with known functions of the Hox family of proteins and suggest potential regulatory mechanisms. For example, Hand2 is structurally related to the Twist gene which has been shown to interact with a domain on HoxA5 (21). As with previous Y2H studies, several of the other interactions detected appear unlikely to be biologically relevant and all putative interactions discovered in such studies require validation by alternative methods.

### Application of the BI-Tag Y2H Technology to High Throughput Protein-Protein Interaction Screening

In the present Examples, we demonstrate the feasibility of library by library screen of mouse proteins for protein-protein interactions where based on the retest fidelity, and that our data is of comparable quality to that of other high-throughput Y2H studies (2). The sample of BI-Tags that were sequenced have a similar level of redundancy to the individually identified colonies suggesting that the BI-Tag procedure is able to accurately represent the members of the original two-hybrid positive library. Furthermore, one third of the individually identified pairs were also identified by BI-Tags. Five out of six of the protein pairs that were individually identified multiple times were also identified by BI-Tag Y2H. Based on these data, we find no obvious bias in the representation of cDNAs identified by the BI-Tag method relative to the interactions defined by traditional YTH methods, which indicates that application of the BI-Tag method to complex libraries is likely to be efficient.

It is expected that the BI-Tag Y2H technology described here can, in conjunction with a variety of HTP parallel sequencing technologies, provide various methods by which to assay a number of interactions sufficient to allow the generation of near comprehensive interaction maps for one or even many mammalian tissues. Further, we expect that generation of bait and prey libraries of a size sufficient to represent near to the total complexity of a cDNA from any given tissue (˜100,000 independent transformants from a normalized cDNA pool) is possible. In addition, methodologies allowing the generation of a sufficient number of yeast zygotes (1×10¹⁰) to assay all of the potential interactions between bait and prey libraries of this size by mating have been described (22). However, a screen of this size could result in as many as 300,000 interactions which, in a standard Y2H approach, would need to be defined by individual sequencing reactions. Nevertheless, certain DNA sequencing technology techniques allow the parallel generation of ˜250,000 unique 100-nucleotide-long sequence reads within a few hours using a single instrument (23), providing the capacity to efficiently generate the requisite amount of sequence data. However, using the standard YTH method associations between interacting bait and prey cDNAs would be lost in this format. The contribution that the BI-Tag technology of the present invention can make is to allow the associations between individual interacting bait and prey sequences to be maintained such that one interaction is represented in each 100-nucleotide long sequence read. Specifically, direct parallel sequencing of the ˜86 by MmeI cleavage products from the linked interacting bait-prey cDNA sequences (as shown schematically in FIG. 8c and in FIG. 9b and omitting the concatenation step), each containing the lox66/71 sequence flanked by MmeI sites and 19-21 by tags that identify interaction partners could identify on the order of 250,000 protein interaction partners per sequencing run. The number of interactions identified from even a few such sequencing runs could, theoretically, allow redundant coverage of all of the potential interactions occurring between the proteins encoded by cDNA derived from a mammalian tissue. From this information a near comprehensive protein interaction map could be established and confidence levels for specific interactions estimated based on repeated representation of any given protein pair. One method by which the sequencing of the ˜86 by MmeI cleavage products could be achieved is by the “massively parallel pyrosequencing” sequencing method, which can be performed by commercially available sequencing services, such as that offered by ROCHE. Massively parallel pyrosequencing is suitable for whole genome sequencing in microfabricated high-density picolitre reactors (23). Other suitable techniques as will be recognized by those skilled in the art can also be used.

### REFERENCES

1. Fields, S, and Song, O. (1989) A novel genetic system to detect protein-protein interactions. Nature 340, 245-246.
2. Uetz, P., Giot, L., Cagney, G., Mansfield, T. A., Judson, R. S., Knight, J. R., Lockshon, D., Narayan, V., Srinivasan, M., Pochart, P., et al. (2000) A comprehensive analysis of protein-protein interactions in Saccharomyces cerevisiae. Nature 403, 623-627.
3. Ito, T., Chiba, T., Ozawa, R., Yoshida, M., Hattori, M., and Sakaki, Y. (2001) A comprehensive two-hybrid analysis to explore the yeast protein interactome. Proc. Natl. Acad. Sci. U.S.A., 98, 4569-4574.
4. Giot, L., Bader, J. S., Brouwer, C., Chaudhuri, A., Kuang, B., Li, Y., Hao, Y. L., Ooi, C. E., Godwin, B., Vitols, E., et al. (2003) A protein interaction map of Drosophila melanogaster. Science, 302, 1727-1736.
5. Li, S., Armstrong, C. M., Bertin, N., Ge, H., Milstein, S., Boxem, M., Vidalain, P. O., Han, J. D., Chesneau, A., Hao, T., et al. (2004) A map of the interactome network of the metazoan C. elegans. Science, 303, 540-543.
6. Stelzl, U., Worm, U., Lalowski, M., Haenig, C., Brembeck, F. H., Goehler, H., Stroedicke, M., Zenkner, M., Schoenherr, A., Koeppen, S., et al. (2005) A human protein-protein interaction network: a resource for annotating the proteome. Cell, 122, 957-968.
7. Rual, J. F., Venkatesan, K., Hao, T., Hirozane-Kishikawa, T., Dricot, A., Li, N., Berriz, G. F., Gibbons, F. D., Dreze, M., Ayivi-Guedehoussou, N., et al. (2005) Towards a proteome-scale map of the human protein-protein interaction network. Nature, 437, 1173-1178.
8. Durfee, T., Draper, O., Zupan, J., Conklin, D. S., and Zambryski, P. C. (1999) New tools for protein linkage mapping and general two-hybrid screening. Yeast, 15, 1761-1768.
9. Pruitt, S. C., Bussman, A., Maslov, A. Y., Natoli, T. A., and Heinaman, R. (2004) Hox/Pbx and Brn binding sites mediate Pax3 expression in vitro and in vivo. Gene Expr. Patterns., 4, 671-685.
10. Feil, R., Wagner, J., Metzger, D., and Chambon, P. (1997) Regulation of Cre recombinase activity by mutated estrogen receptor ligand-binding domains. Biochem. Biophys. Res. Commun., 237, 752-757.
11. Wach, A., Brachat, A., Pohlmann, R., and Philippsen, P. (1994) New heterologous modules for classical or PCR-based gene disruptions in Saccharomyces cerevisiae. Yeast, 10, 1793-1808.
12. Sauer, B. and Henderson, N. (1990) Targeted insertion of exogenous DNA into the eukaryotic genome by the Cre recombinase. New Biol., 2, 441-449.
13. Cheng, T. H., Chang, C. R., Joy, P., Yablok, S., and Gartenberg, M. R. (2000) Controlling gene expression in yeast by inducible site-specific recombination. Nucleic Acids Res., 28, e108.
14. Hoffman, C. S. and Winston, F. (1987) A ten-minute DNA preparation from yeast efficiently releases autonomous plasmids for transformation of Escherichia coli. Gene, 57, 267-272.
15. Albert, H., Dale, E. C., Lee, E., and Ow, D. W. (1995) Site-specific integration of DNA into wild-type and mutant lox sites placed in the plant genome. Plant J., 7 , 649-659.
16. Abremski, K., Hoess, R., and Sternberg, N. (1983) Studies on the properties of P1 site-specific recombination: evidence for topologically unlinked products following recombination. Cell, 32, 1301-1311.
17. Fromont-Racine, M., Rain, J. C., Legrain, P. (1997) Toward a functional analysis of the yeast genome through exhaustive two-hybrid screens. Nat. Genet., 16(3):277-82.
18. Rain, J. C., Selig, L., De Reuse, H., Battaglia, V., Reverdy, C., Simon, S., Lenzen, G., Petel, F., Wojcik, J., Schachter, V., Chemama, Y., Labigne, A., and Legrain, P. (2001) The protein-protein interaction map of Helicobacter pylori. Nature, 409, 211-215.
19. Muratani, M. and Tansey, W. P. (2003) How the ubiquitin-proteasome system controls transcription. Nat. Rev. Mol. Cell. Biol., 4, 192-201.
20. Citterio, E., Papait, R., Nicassio, F., Vecchi, M., Gomiero, P., Mantovani, R., Di Fiore, P. P., and Bonapace, I. M. (2004) Np95 is a histone-binding protein endowed with ubiquitin ligase activity. Mol. Cell. Biol., 24, 2526-2535.
21. Stasinopoulos, I. A., Mironchik, Y., Raman, A., Wildes, F., Winnard, P., Jr., and Raman, V. (2005) HOXA5-twist interaction alters p53 homeostasis in breast cancer cells. J. Biol. Chem., 280, 2294-2299.
22. Soellick, T. R. and Uhrig, J. F. (2001) Development of an optimized interaction-mating protocol for large-scale yeast two-hybrid analyses. Genome Biol., 2, RESEARCH0052.
23. Margulies, M., Egholm, M., Altman, W. E., Attiya, S., Bader, J. S., Bemben, L. A., Berka, J., Braverman, M. S., Chen, Y. J., Chen, Z., et al. (2005) Genome sequencing in microfabricated high-density picolitre reactors. Nature, 437, 376-380.

What is claimed is:

1. A method for identifying a plurality of pairs of interacting proteins wherein a pair of interacting proteins comprises a first test protein and a second test protein, wherein the first and second test proteins interact with each other in a cell, the method comprising the steps of:
a) providing a cDNA library;
b) providing a plurality of a first plasmid comprising a coding sequence for a
DNA binding domain of a transcription activator, a first recombinase recognition site, a first selectable marker, a first Type II S restriction site and a first inserted cDNA encoding a first test protein;
c) providing a plurality of a second plasmid comprising a coding sequence for a transcription activation domain of the transcription activator, a second recombinase recognition site, a second selectable marker and a second Type II S restriction site, and a second inserted cDNA encoding a second test protein, wherein the first and second recombinase recognition sites may be identical or distinct and the first and second Type II S restriction sites may be identical or distinct;
d) introducing the first and second plasmids from b) and c) into the same cell;
e) inducing the expression of the recombinase to recombine the first and second introduced plasmids;
f) isolating and digesting the recombined plasmids with a Type II S restriction enzyme to obtain a plurality of restriction fragments, and
g) determining the sequence of the plurality of restriction fragments to determine the identity of the plurality of pairs of interacting proteins.

2. The method of claim 1, wherein the first and second inserted cDNAs of steps
b) and c) are inserted by homologously recombining the first and second cDNAs with the first and second plasmids, respectively.

3. The method of claim 1, wherein the Type II S restriction site is selected from the group consisting of BsgI, BpmI, and MmeI sites.

4. The method of claim 1, wherein the recombinase recognition sites are half mutant sites.

5. The method of claim 1 wherein step d) comprises introducing the first and second plasmids into the same cell by mating a first and second yeast cell, wherein the first yeast cell has been transformed with either the first or second plasmid, and wherein the second yeast cell has been transformed with the first or second plasmid with which the first yeast cell was not transformed.

6. The method of claim 1, wherein in step e) the cell into which the first and second plasmids are introduced is selected for by interaction of proteins encoded by the first and second cDNAs, wherein the interaction induces expression of a selectable marker, wherein expression of the selectable marker permits the cell to survive or to be distinguished from cells not expressing the selectable marker.

7. The method of claim 1, wherein step d) is performed by massively parallel pyrosequencing.

8. A plasmid comprising a recombinase recognition site, a cloning site for cloning a cDNA into the plasmid, at least one selectable marker, a Type II S restriction site, and a coding sequence, wherein the coding sequence is selected from the group consisting of:
a) a coding sequence for a DNA binding domain of a transcription activator such that the DNA binding domain of the transcription activator can be expressed as a fusion protein with the protein encoded by the cDNA; and
b) a coding sequence for a transcription activation domain of a transcription activator such that the DNA transcription activation domain of the transcription activator can be expressed as a fusion protein with the protein encoded by the cDNA.

9. The plasmid of claim 8, wherein the recombinase recognition site is recognized by a recombinase selected from the group consisting of Cre recombinase, tamoxefin inducible Cre recombinase, and FLP recombinase.

10. The plasmid of claim 8, wherein the transcription activator is Gal4.

11. The plasmid of claim 8, wherein the Type II S restriction site is selected from the group consisting of BsgI, BpmI, and MmeI sites.

12. The plasmid of claim 8, wherein the recombinase recognition sites are half mutant sites.

13. The recombinase recognition sites of claim 12, wherein the sites are selected from lox71 and lox66 sites.

14. The plasmid of claim 8, wherein the selectable marker is selected from the group consisting of LEU2, URA3, HIS3, TRP1, ADE2 and LYS2.

15. A kit for determining interacting proteins, wherein the kit comprises:
a) a first plasmid comprising a coding sequence for a DNA binding domain of a transcription activator, a cloning site for cloning a first cDNA into the first plasmid such that the DNA binding domain of the transcription activator can be expressed as a fusion protein with the protein encoded by the first cDNA, a first recombinase recognition site, a first selectable marker, and a first Type II S restriction site; and
b) a second plasmid comprising a coding sequence for a transcription activation domain of the transcription activator, the cloning site for cloning a second cDNA into the second plasmid such that the transcription activation domain of the transcription activator can be expressed as a fusion protein with the protein encoded by the second cDNA, a second recombinase recognition site, a second selectable marker, and a second Type II S restriction site, wherein the first and second recombinase recognition sites may be identical or distinct and the first and second Type II S restriction sites may be identical or distinct.

16. The first and second plasmids of claim 15, wherein the first and second recombinase recognition sites are recognized by a recombinase selected from the group consisting of Cre recombinase, tamoxefin inducible Cre recombinase, and FLP recombinase.

17. The first and second plasmids of claim 15, wherein the transcription activator is Gal4.

18. The first and second plasmids of claim 16, wherein the first and second Type II S restriction enzymes are selected from the group consisting of BsgI, BpmI, and MmeI.

19. The first and second plasmids of claim 16, wherein the first and second recombinase recognition sites are half mutant sites.

20. The first and second plasmids of claim 16, wherein the first plasmid has either the first recombinase recognition site lox71 or lox66, and wherein the second plasmid has a second recombinase recognition site selected from lox71 or lox66, wherein the second recombinase recognition site is the site the first plasmid does not have.


SEQUENCE LISTING (38 sequences)

SEQ ID NO: 1 - DNA, 48 bp, Artificial Sequence
  n 43,44, 45, 46, 47, 48: n is g,a,t or c;
  pAct2 lox71 MAGE/6 Primer
gtatagcatacattatacgaacggtaaccctctgagctggagnnnnnn

SEQ ID NO: 2 - DNA, 48 bp, artificial sequence
  n 43-48: n is g,a,t or c
  PCD2 lox66 MAGE/6 Primer
cgtataatgtatgctatacgaacggtaccctctgagctggagnnnnnn

SEQ ID NO: 3 - DNA, 40 bp, artificial sequence
  n 35-40: n is g,a,t or c
  Lox71 MmeI primer
tataatgtatgctatacgaacggtaggatccaacnnnnnn

SEQ ID NO: 4 - DNA, 38 bp, artificial sequence
  n 33-38: n is g,a,t or c
  Lox66 MmeI primer
catatcgtatgtaatatgcttgccataggttgnnnnnn

SEQ ID NO: 5 - DNA, 529 bp, artificial sequence
  n 392, 448, 453, 455, 473, 476, 479, 505,522, 524, 528: n is a, g, t, c
  concatamerized sequence tags
atcccccgggctgcaggaattcgatgcgataataaccacggccaccactggagggatcccttgatcagacaccactggagcacgagaagaaggagccaccactggagcacgagaagaaggagctcaccactggagggatcccttgatcagacaccactggagggggtcgggacggagacaccactggaggagggcacagcagaagcaccactggagggtggggactttctcccaccactggagggatcccttgatcatacaccactggagagggtcccgatgctggcaccactggagcctcgatcagatctgccaccactggagcactagaaaaagaggacaccactggaggagggcacagcagaagcaccactggagggtggggactttcntcccaccactggagtgctcgttagaatattcaccactggagggatcccttgatcanacacntnctggagcggacagagganacntcnaccactggagcggcaggggaacttanccccacttgggaccacnanaagna

SEQ ID NO: 6 - DNA, 16 bp, mouse
  mouse cDNA sequence tag
cgataataaccacggc

SEQ ID NO: 7 - DNA, 16 bp, mouse
  mouse cDNA sequence tag
ggatcccttgatcaga

SEQ ID NO: 8 - DNA, 16 bp, mouse
  mouse cDNA sequence tag
cacgagaagaaggagc

SEQ ID NO: 9 - DNA, 17 bp, mouse
  mouse cDNA sequence tag
cacgagaagaaggagct

SEQ ID NO: 10 - DNA, 16 bp, mouse
  mouse cDNA sequence tags
ggatcccttgatcaga

SEQ ID NO: 11 - DNA, 16 bp, mouse
  mouse cDNA sequence tag
ggggtcgggacggaga

SEQ ID NO: 12 - DNA, 16 bp, mouse
  mouse cDNA sequence tag
gagggcacagcagaag

SEQ ID NO: 13 - DNA, 16 bp, mouse
  mouse cDNA sequence tag
ggtggggactttctcc

SEQ ID NO: 14 - DNA, 16 bp, mouse
  mouse cDNA sequence tag
ggatcccttgatcata

SEQ ID NO: 15 - DNA, 16 bp, mouse
  mouse cDNA sequence tag
agggtcccgatgctgg

SEQ ID NO: 16 - DNA, 16 bp, mouse
  mouse cDNA sequence tag
cctcgatcagatctgc

SEQ ID NO: 17 - DNA, 16 bp, mouse
  mouse cDNA sequence tag
cactagaaaaagagga

SEQ ID NO: 18 - DNA, 16 bp, mouse
  mouse cDNA sequence tag
gagggcacagcagaag

SEQ ID NO: 19 - DNA, 17 bp, mouse
  n 14: n is g,t,a or c; mouse cDNA sequence tag
ggtggggactttcntcc

SEQ ID NO: 20 - DNA, 16 bp, mouse
  mouse cDNA sequence tag
tgctcgttagaatatt

SEQ ID NO: 21 - DNA, 16 bp, mouse
  n 15: n is g,t,a or c; mouse cDNA sequence tag
ggatcccttgatcana

SEQ ID NO: 22 - DNA, 16 bp, mouse
  n 12, 15: n is g,t,a or c; mouse cDNA sequence tag
cggacagagganacnt

SEQ ID NO: 23 - DNA, 16 bp, mouse
  n 16: n is g,t,a or c; mouse cDNA sequence tags
cggcaggggaacttan

SEQ ID NO: 24 - DNA, 16 bp, mouse
gcagatctgatcgagg

SEQ ID NO: 25 - DNA, 39 bp, mouse
tggccatggacctaggcagatctgatcaagggatccggg

SEQ ID NO: 26 - DNA, 52 bp, artificial sequence
  n 47, 48, 49, 50, 51, 52: n is g, t, a or c
  CDS-52 lox71 sequence
gctgcagataacttcgtataatgtatgctatacgaacggtatccaacnnnnn

SEQ ID NO: 27 - DNA, 47 bp, mouse
  n 48, 49, 50, 51, 52: n is g, t, a or c
  SMART pGBK47
gagcagaagctgatctcagaggaggacctgcatatggccatggaggg

SEQ ID NO: 28 - DNA, 54 bp, artificial sequence
  n 50, 51, 52, 53, 54: n is g, t, a or c
  CDS-54 lox66
ggctgcagcataacttcgtatagcatacattatacgaacggtatccaacnnnnn

SEQ ID NO: 29 - DNA, 38 bp, artificial sequence
  n 37, 38: n is g, t, a or c
  NotI linker t3
gcgggatagcgtgccagcgagtgacgttgcggccgcnn

SEQ ID NO: 30 - DNA, 36 bp, artificial sequence
  NotI linker b3
gcggccgcaacgtcactcgctggcacgctatcccgc

SEQ ID NO: 31 - DNA, 40 bp, artificial sequence
  n 39, 40: n is g, t, a or c
  NotI linker t4
ggtatagcccggcagttgcgctgacgagcagcggccgcnn

SEQ ID NO: 32 - DNA, 38 bp, artificial sequence
  NotI linker b4
gcggccgctgctcgtcagcgcaactgccgggctatacc

SEQ ID NO: 33 - DNA, 36 bp, artificial sequence
  sequence used in cloning strategy to produce yeast-two-hybrid libraries
tggccatggacctaggcagatctgatcaagggatcc

SEQ ID NO: 34 - DNA, 40 bp, artificial sequence
  sequence used in cloning strategy to produce yeast-two-hybrid libraries
gttggataccgttcgtatagcatacattatacgaagttat

SEQ ID NO: 35 - DNA, 36 bp, artificial sequence; identical to SEQ ID NO: 33
  sequence used in cloning strategy to produce yeast-two-hybrid libraries

SEQ ID NO: 36 - DNA, 40 bp, artificial sequence
  sequence used in cloning strategy to produce yeast-two-hybrid libraries
gttggataccgttcgtataatgtatgctatacgaagttat

SEQ ID NO: 37 - DNA, 37 bp, artificial sequence
  sequence used in cloning strategy to produce yeast-two-hybrid libraries
tgatctcagaggaggacctgcatatggccatggaggg

SEQ ID NO: 38 - DNA, 40 bp, artificial sequence; identical to SEQ ID NO: 36
  sequence used in cloning strategy to produce yeast-two-hybrid libraries